(19) United States
(12) Patent Application Publication
(10) Pub. No.: US 2003/0133927 A1
(43) Pub. Date: Jul. 17, 2003

(54) Conjugates useful in the treatment of prostate cancer

(76) Inventors: Deborah DeFeo-Jones, Lansdale, PA (US); Raymond E. Jones, Lansdale, PA (US)

Correspondence Address: MERCK AND CO INC, P O BOX 2000, RAHWAY, NJ, 070650907

(21) Appl. No.: 10/268,552

(22) Filed: Oct. 10, 2002

Related U.S. Application Data
(60) Provisional application No. 60/328,351, filed on Oct. 10, 2001.

(51) Int. Cl. A61K 38/48; C12N 9/99

(52) U.S. Cl. 424/94.63; 435/184

(57) ABSTRACT

Chemical conjugates which comprise an oligopeptide covalently bonded, either directly or through a chemical linker, to a peptide or small molecule that binds to an anti-apoptotic Bcl-2 family protein, inhibits the expression of the Bcl-2 family protein, or inhibits the function of the Bcl-2 family protein. Such a peptide or small molecule that binds to an anti-apoptotic Bcl-2 family protein, inhibits the expression of the Bcl-2 family protein, or inhibits the function of the Bcl-2 family protein may be conveniently referred to as a therapeutic agent. The oligopeptides are chosen from oligomers that are selectively recognized by the free prostate specific antigen (PSA) and are capable of being proteolytically cleaved by the enzymatic activity of the free prostate specific antigen.

### BACKGROUND OF THE INVENTION

In 1999 new cases of cancer of the prostate gland were expected to be diagnosed in 179,300 men in the U.S. and 37,000 American males were expected to die from this disease (Landis, S. H. et al. CA Cancer J. Clin. 49:8-31 (1999)). Prostate cancer is the most frequently diagnosed malignancy (other than that of the skin) in U.S. men and the second leading cause of cancer-related deaths (behind lung cancer) in that group.

Prostate specific Antigen (PSA) is a single chain 33 kDa glycoprotein that is produced almost exclusively by the human prostate epithelium and occurs at levels of 0.5 to 2.0 mg/ml in human seminal fluid (Nadji, M., Taber, S. Z., Castro, A., et al. (1981) Cancer 48:1229; Papsidero, L., Kuriyama, M., Wang, M., et al. (1981). JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979). Invest. Urol. 17:159). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985). J. Clin. Invest. 76:1899; Lilja, H., Oldbring, J., Rannevik, G., et al. (1987). J. Clin. Invest. 80:281; McGee, R. S., Herr, J. C. (1988). Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatoza (Lilja, H., Laurell, C. B. (1984). Scand. J. Clin. Lab. Invest. 44:447; McGee, R. S., Herr, J. C. (1987). Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., Björk, T., Nilsson, O., et al. (1993). J. Urol. 150:100-105; Lilja, H., Christensson, A., Dahlén, U. (1991). Clin. Chem. 37:1618-1625; Stenman, U. H., Leinoven, J., Alfthan, H., et al. (1991). Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991). Biochemistry 30:1723-1730; Perlmutter, D. H., Glover, G. I., Rivetna, M., et al. (1990). Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule.

Serum measurements of PSA are useful for monitoring the treatment of adenocarcinoma of the prostate (Duffy, M. S. (1989). Ann. Clin. Biochem. 26:379-387; Brawer, M. K. and Lange, P. H. (1989). Urol. Suppl. 5:11-16; Hara, M. and Kimura, H. (1989). J. Lab. Clin. Med. 113:541-548), although above normal serum concentrations of PSA have also been reported in benign prostatic hyperplasia and subsequent to surgical trauma of the prostate (Lilja, H., Christensson, A., Dahlén, U. (1991). Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985). Brit. J. Urology 57:50-55). Therefore, a cytotoxic compound that could be activated by the proteolytic activity of PSA should be prostate cell and prostate cancer cell specific as well as specific for PSA secreting prostate cancer metastases.

U.S. Pat. No. 4,203,898 describes derivative of the vinca alkaloid cytotoxic agents wherein the C-3 methyl ester of the vinca drug has been modified.

Bcl-2 (B cell lymphoma/leukemia 2) was originally identified at the chromosomal breakpoint of t(14; 18)-bearing B-cell lymphomas. Bcl-2 is now known to belong to a growing family of proteins which regulate programmed cell death or apoptosis. The Bcl-2 family includes both death antagonists (Bcl-2, Bcl-x_{L}, Bcl-w, Bfl-1, Brag-I, Mcl-I and Al) and death agonists (Bax, Bak, Bcl-X5, Bad, Bid, Bik and Hrk) (Thompson, Science 267:1456-62 (1992); Reed, J. Cell Biol. 124:1-6 (1994); Yang et al., Blood 88:386-401 (1996)). This family of molecules shares four homologous regions termed Bcl homology (BH) domains BH1, BH2, BH3, and BH4. All death antagonist members contain the BH4 domain while the agonist members lack BH4. It is known that the BH1, BH2 and BH3 domains of the death antagonists such as Bcl-2 are required for these proteins to heterodimerize with death agonists, such as Bax, and to repress cell death. On the other hand, the BH3 domain of death agonists is required for these proteins to heterodimerize with Bcl-2 and to promote apoptosis.

Programmed cell death or apoptosis plays a fundamental role in the development and maintenance of cellular homeostasis. Homologous proteins and pathways in apoptosis are found in a wide range of species, indicating that cellular demise is critical for the life and death cycle of the cell in all organisms. When extracellular stimuli switch on the cell-death signal, the response of the cell to such stimuli is specific for the particular cell type (Bonini et al., Cell 72:379-95 (1993)). The pathway to cellular suicide is controlled by certain checkpoints (Oltvai, Cell 79:189-92 (1994)). The Bcl family proteins, including both antagonists of apoptosis (such as Bcl-2) and agonists of apoptosis (such as Bax), constitute the primary checkpoint. As such, the transmission of a cell-death signal can be either promoted or blocked by the different combinations of the Bcl-2 family members. The three-dimensional structure of a death antagonist, Bcl-x_{L}, as determined by X-ray crystallography and NMR spectroscopy, provides a structural basis for understanding the biological functions of Bcl-2 family members and for developing novel therapeutics targeting Bcl-2 mediated apoptatic pathways (Muchmore et al., Nature 381:335-41 (1996)).

The detailed mechanism of Bcl-2 proteins in mediating molecular pathways of apoptosis has been the subject of intensive investigation. It is known that the apoptotic signaling pathway involves the activation of caspases which, once activated, cleave several cellular substrates such as poly(adenosine diphosphateribose) polymerase (PARP) and lead to final events of apoptosis. Bcl-2 plays a crucial role in regulating the process of apoptosis. One possible mechanism for Bcl-2 function is that Bcl-2 sequesters proapoptotic proteins thereby inhibiting the release of cytochrome c from mitochondria. Cytochrome c is important for the activation of caspases, which leads to apoptosis. As such, Bcl-2 blocks caspase activation and subsequent events leading to apoptosis.

Being able to block apoptosis, Bcl-2 is known to contribute to neoplastic cell expansion by preventing normal cell turnover caused by physiological cell death mechanisms. High levels and aberrant patterns of Bcl-2 gene expression are found in a wide variety of human cancers, including ˜30-60% of prostate, ˜90% of colorectal, ˜60% of gastric, ˜20% of non-small cell lung cancers, ˜30% of neuroblastomas, and variable percentages of melanomas, renal cell, and thyroid cancers, as well as acute and chronic lymphocytic and non-lymphocytic leukemias (Ellis at a/., Cell Biol. 7, 663 (1991); Henkart, Immunity 1, 343 (1994)); Kagi at al., Science 265, 528 (1994); Kagi et al., Nature 369, 31 (1994); Heusel et al., Cell 76, 977 (1994)).

The expression levels of Bcl-2 protein also correlate with relative resistance to a wide spectrum of current chemotherapeutic drugs and γ-irradiation (Hanada et al, Cancer Res. 53:4978-86 (1993); Kitada et al., Antisense Res. Dev. 4:71-9 (1994); Miyashita et al., Cancer Res. 52:5407-11 (1992); Miyashita at al., Blood 81:151-7 (1993)). Since Bcl-2 can protect against such a wide variety of drugs that have very different mechanisms of action, it is possible that all these drugs use a common final pathway for the eventual induction of cell death which is regulated by Bcl-2. This notion is supported by the findings that chemotherapeutic drugs induce cell death through a mechanism consistent with apoptosis as opposed to necrosis. Therefore, Bcl-2 can inhibit the cell killing effect of currently available anticancer drugs by blocking the apoptotic pathway.

Because of its role in blocking apoptosis, Bcl-2 plays an important role in many types of cancer. As noted above, Bcl-2 blocks apoptosis, thereby preventing normal cell turnover. As a result, neoplastic cell expansion occurs unimpeded by the normal cellular turnover process. Prostate cancer is one particular example where Bcl-2 has important implication in the pathogenesis and treatment for a disease. Approximately 100,000 new cases of prostate cancer are diagnosed each year in the United States and about 30,000 deaths per year are attributable to this disease (Lynn et al., JNCI 87:867 (1995)). It has recently been found that hormone therapy-resistant prostate cancers express Bcl-2 (McDonnell et al. Cancer Res. 52:6940-4 (1992)), while the normal prostate cells from which prostate cancers originate lack BcI-2 (Colombel et al, Am J Pathol 143:390-400 (1993)). This indicates that Bcl-2 may protect prostate cancer cells from undergoing apoptosis induced by the anticancer drugs, such as Taxol (Haldar et al, Cancer Res., 56:1235-5 (1996)). The clinical efficacy of nearly every cytotoxic anticancer drug currently available depends directly or indirectly on the assumption that tumor cells grow more rapidly than normal cells. However, this may not apply to human prostate cancer cells, which show very slow growth kinetics. Tumor kinetics studies have indicated that prostate cancer may be the consequence of the imbalance in cell turnover mechanisms more so than an increase in cell cycle rates. Thus, current anticancer drugs may not be effective in eradicating these nonproliferative prostate cancer cells.

The understanding of the biology of Bcl-2 in cancer and chemoresistance has opened new avenues in the development of novel anticancer strategies. One effective approach to overcome the chemoresistance of prostate cancers is to inhibit the protective function of Bcl-2 proteins. New drugs that modulate Bcl-2 mediated apoptotic response would represent a novel mechanism-based strategy for the treatment of prostate cancers and other cancers. Because the function of Bcl-2 is not absolutely necessary in many normal cell types (Veis et al, Cell, 75:229-40 (1993)), a systematic inhibition of Bcl-2 may not affect the normal cellular function. This notion is supported by recent encouraging data from the clinical trial that antisense oligonucleotides targeted against the Bcl-2 gene can specifically inhibit non-Hodgkin's lymphoma in humans (Webb at al, Lancet 349,1137-41 (1997)). However, the clinical value of such antisense oligonucleotides is limited by their lack of stability, cell permeability, and oral activity. As discussed above, currently available anticancer drugs may not be effective due to the chemoresistance of prostate cancer cells. Therefore, there is an impending need for highly potent, cell permeable, and orally active Bcl-2 inhibitors as a new generation of effective therapeutics for the treatment of prostate cancer, as well as other cancers.

Amarante-Mendes et al., Oncogene, 1998, 16, 1383-1390, disclose antisense oligonucleotides targeted to bcr and bcl-x. The latter downregulated the expression of bcl-x_{L} and increased the susceptibility of HL-60 Bcr-Abl cells to staurosporine.

U.S. Pat. No. 5,583,034 (Green et al.) discloses antisense oligonucleotides which hybridize to the nucleic acid sequence of an anti-apoptotic gene, preferably to the translation start site of bcr-abl. Wang et al. used a phosphorothioate oligonucleotide targeted to the bcl-x translation start site to block CD40L mediated apoptotic rescue in murine WEHI-231 lymphoma cells (J. Immunol., 1995, 155, 3722-3725). Fujio et al. have used an antisense oligodeoxynucleotide targeted to murine and rat bcl-x mRNA to reduce bcl-x_{L} protein expression (J. Clin. Invest., 1997, 99, 2898-2905). The compound tested was the same as that of Wang et al. Oligonucleotide treatment inhibited the cytoprotective effect of leukemia inhibitory factor in mouse or rat cardiac myocytes. Pollman et al. used antisense oligodeoxynucleotides with phosphorothioate backbones to downregulate bcl-x_{L} expression in blood vessel intimal cells (Nature Med., 1998, 4, 222-227). This resulted in induction of apoptosis and regression of vascular lesions. Antisense sequences were targeted to the translation initiation codon of mouse/human bcl-x (conserved sequence) and were used in rabbits. Gibbons et al., U.S. Pat. No. 5,776,905, disclose methods for targeted deletion of intimal lesion cells in the vasculature of a mammal with vascular disease, preferably with antisense molecules specific for anti-apoptotic genes, more preferably bcl-x and most preferably bcl-x_{L}. Thompson et al., U.S. Pat. No. 5,646,008 and WO 95/00642 describe an isolated and purified polynucleotide that encodes a polypeptide other than Bcl-2 that promotes or inhibits programmed vertebrate cell death. Preferably the polypeptide is Bcl-x_{L}, Bcl-xs or Bcl-xₗ. Polypeptides, polynucleotides identical or complementary to a portion of the isolated and purified polynucleotide, expression vectors, host cells, antibodies and therapeutic and diagnostic methods of use are also provided. Yang et al., WO 98/05777 disclose Bcl-xγ (gamma), a novel isoform of the bcl-x family which includes an ankyrin domain. Polypeptide and nucleic acid sequences, for this isoform are disclosed, as well as, inter alia, methods for modulating Bcl-xγ activity, including antisense methods.

It is the object of this invention to provide a novel anti-cancer composition useful for the treatment of prostate cancer which comprises oligopeptides, that are selectively proteolytically cleaved by free prostate specific antigen (PSA) and that are linked to a peptide or small molecule that binds to an anti-apoptotic BCL-2 family protein.

Another object of this invention is to provide a method of treating prostate cancer that comprises administration of the novel anti-cancer composition.

### SUMMARY OF THE INVENTION

Chemical conjugates which comprise oligopeptides, having amino acid sequences that are selectively proteolytically cleaved by enzymatically active prostate specific antigen (PSA), and a peptide, oligonucleotide or small molecule that inhibits the function of a Bcl-2 family protein are disclosed. Such conjugates are useful in the treatment of pro static cancer and benign prostatic hyperplasia (BPH).

### DETAILED DESCRIPTION OF THE INVENTION

The instant invention relates to novel anti-cancer compositions useful for the treatment of prostate cancer. Such compositions comprise an oligopeptide covalently bonded, either directly or through a chemical linker, to a peptide, oligonucleotide or small molecule that inhibits the anti-apoptotic function of a Bcl-2 family protein. Such a peptide, oligonucleotide or small molecule that binds to an anti-apoptotic Bcl-2 family protein, inhibits the expression of such a Bcl-2 family protein or inhibits the function of such a Bcl-2 family protein in another way, may be conveniently referred to as a therapeutic agent. The oligopeptides are chosen from oligomers that are selectively recognized by the enzymatically active prostate specific antigen (PSA) and are capable of being proteolytically cleaved by the enzymatic activity of the prostate specific antigen. Such a combination of an oligopeptide and therapeutic agent may be termed a conjugate.

As used herein, the “function of a Bcl-2 family protein” is understood to be the sequestering of pro-apoptotic proteins (such as Bad, Bax and the like). Therapeutic agents useful as components in the instant conjugates may inhibit such function by binding to an anti-apoptotic Bcl-2 family protein, by inhibiting the expression of such a Bcl-2 family protein or by competitively binding to the pro-apoptotic proteins that the Bcl-2 protein binds to, thereby preventing the association of the anti-apoptotic protein to a pro-apoptotic protein. Other methods for inhibiting the function of an anti-apoptotic Bcl-2 family protein are also contemplated.

Ideally, the binding and/or inhibitory activity of the therapeutic agent is greatly reduced or absent when the oligopeptide containing the PSA proteolytic cleavage site is bonded, either covalently or through a chemical linker, to the therapeutic agent and is intact. Also ideally, the binding and/or inhibitory activity of the therapeutic agent increases significantly or returns to the activity of the unmodified therapeutic agent upon proteolytic cleavage of the attached oligopeptide at the cleavage site.

If the oligopeptide is attached to the therapeutic agent through a chemical linker, preferably, the therapeutic agent with a chemical linker intact exhibits binding and/or inhibitory activity that is at least 75% of the activity of the unmodified therapeutic agent against the target cancer cells. Such a derivative of the therapeutic agent wherein the chemical linker is still covalently bound to the therapeutic agent may itself be considered a therapeutic agent.

Furthermore, it is preferred that the oligopeptide is selected from oligopeptides that are not cleaved or are cleaved at a much slower rate in the presence of non-PSA proteolytic enzymes, such as those enzymes endogenous to human serum, when compared to the cleavage of the oligopeptides in the presence of free enzymatically active PSA.

For the reasons above, it is desirable for the oligopeptide to comprise a short peptide sequence, preferably less than ten amino acids. Most preferably the oligopeptide comprises seven or six amino acids. Because the conjugate preferably comprises a short amino acid sequence, the solubility of the conjugate may be influenced to a greater extent by the generally hydrophobic character of the therapeutic agent component. Therefore, amino acids with hydrophilic substituents may be incorporated in the oligopeptide sequence or N-terminus blocking groups may be selected to offset or diminish such a hydrophobic contribution by the therapeutic agent. Combinations of amino acids with hydrophilic substituents and N-terminus blocking groups that enhance solubility may also be employed in a single conjugate.

While it is not necessary for practicing this aspect of the invention, an embodiment of this invention is a conjugate wherein the oligopeptide and the chemical linker are detached from the therapeutic agent by the proteolytic activity of the free PSA and any other native proteolytic enzymes present in the tissue proximity, thereby presenting the therapeutic agent, or a therapeutic agent that retains part of the oligopeptide/linker unit but binding and/or inhibitory activity, into the physiological environment at the place of proteolytic cleavage. Pharmaceutically acceptable salts of the conjugates are also included.

It is understood that the oligopeptide, that is conjugated to the therapeutic agent through a chemical linker, does not need to be the oligopeptide that has the greatest recognition by free PSA and is most readily proteolytically cleaved by free PSA. Thus, the oligopeptide that is selected for incorporation in such an anti-cancer composition will be chosen both for its selective, proteolytic cleavage by free PSA and for the binding and/or inhibitory activity of the therapeutic agent-proteolytic residue conjugate (or, in what is felt to be an ideal situation, the unmodified therapeutic agent) which results from such a cleavage. The term “selective” as used in connection with the proteolytic PSA cleavage means a greater rate of cleavage of an oligopeptide component of the instant invention by free PSA relative to cleavage of an oligopeptide which comprises a random sequence of amino acids. Therefore, the oligopeptide component of the instant invention is a prefered substrate of free PSA. The term “selective” also indicates that the oligopeptide is proteolytically cleaved by free PSA between two specific amino acids in the oligopeptide.

The oligopeptide components of the instant invention are selectively recognized by the free prostate specific antigen (PSA) and are capable of being proteolytically cleaved by the enzymatic activity of the free prostate specific antigen. In an embodiment, such oligopeptides comprise an amino acid sequence selected from:

a) AsnLysIleSerTyrGln|Ser (SEQ.ID.NO.: 1),

b) LysIleSerTyrGln|Ser (SEQ.ID.NO.: 2),

c) AsnLysIleSerTyrTyr|Ser (SEQ.ID.NO.: 3),

d) AsnLysAlaSerTyrGln|Ser (SEQ.ID.NO.: 4),

e) SerTyrGln|SerSer (SEQ.ID.NO.: 5);

f) LysTyrGln|SerSer (SEQ.ID.NO.: 6);

g) hArgTyrGln|SerSer (SEQ.ID.NO.: 7);

h) hArgChaGln|SerSer (SEQ.ID.NO.: 8);

i) TyrGln|SerSer (SEQ.ID.NO.: 9);

j) TyrGln|SerLeu (SEQ.ID.NO.: 10);

k) TyrGln|SerNle (SEQ.ID.NO.: 11);

l) ChgGln|SerLeu (SEQ.ID.NO.: 12);

m) ChgGln|SerNle (SEQ.ID.NO.: 13);

n) SerTyrGln|Ser (SEQ.ID.NO.: 14);

o) SerChgGln|Ser (SEQ.ID.NO.: 15);

p) SerTyrGln|SerVal (SEQ.ID.NO.: 16);

q) SerChgGln|SerVal (SEQ.ID.NO.: 17);

r) SerTyrGln|SerLeu (SEQ.ID.NO.: 18);

s) SerChgGln|SerLeu (SEQ.ID.NO.: 19);

t) HaaXaaSerTyrGln|Ser (SEQ.ID.NO.: 20);

u) HaaXaaLysTyrGln|Ser (SEQ.ID.NO.: 21);

v) HaaXaahArgTyrGln|Ser (SEQ.ID.NO.: 22);

w) HaaXaahArgChaGln|Ser (SEQ.ID.NO.: 23);

x) HaaTyrGln|Ser (SEQ.ID.NO.: 24);

y) HaaXaaSerChgGln|Ser (SEQ.ID.NO.: 25);

z) HaaChgGln|Ser (SEQ.ID.NO.: 26);

aa) SerChgGln|SerSer (SEQ.ID.NO.: 27);

bb) SerChgGln|SerPro (SEQ.ID.NO.: 28);

cc) SerChgGln|SerAbu (SEQ.ID.NO.: 29);

wherein Haa is a cyclic amino acid substituted with a hydrophilic moiety, hArg is homoarginine, Xaa is any amino acid, Cha is cyclohexylalanine, Abu is 2-aminobutyric acid and Chg is cyclohexylglycine.

In another embodiment of the instant invention, the oligopeptide comprises an amino acid sequence selected from:

a) SerSerTyrGln|SerVal (SEQ.ID.NO.: 30);

b) SerSerChgGln|SerVal (SEQ.ID.NO.: 31);

c) SerSerTyrGln|SerLeu (SEQ.ID.NO.: 32);

e) SerSerChgGln|SerLeu (SEQ.ID.NO.: 33);

f) SerSerTyrGIn|SerSer (SEQ.ID.NO.: 34);

g) SerSerChgGln|SerSer (SEQ.ID.NO.: 35);

h) SerSerTyrGln|SerPro (SEQ.ID.NO.: 36);

i) SerSerChgGln|SerPro (SEQ.ID.NO.: 37);

j) 4-HypSerSerTyrGln|Ser (SEQ.ID.NO.: 38);

k) 4-HypSerSerChgGln|Ser (SEQ.ID.NO.: 39);

l) AlaSerTyrGln|SerVal (SEQ.ID.NO.: 40);

m) AlaSerChgGln|SerVal (SEQ.ID.NO.: 41);

n) AlaSerTyrGln|SerLeu (SEQ.ID.NO.: 42);

o) AlaSerChgGln|SerLeu (SEQ.ID.NO.: 43);

p) 4-HypAlaSerTyrGln|Ser (SEQ.ID.NO.: 44);

q) 4-HypAlaSerChgGln|Ser (SEQ.ID.NO.: 45);

wherein 4-Hyp is 4-hydroxyproline, and Chg is cyclohexylglycine.

In a more preferred embodiment of the instant invention, the oligopeptide comprises an amino acid sequence selected from:

SerSerChgGln|SerLeu (SEQ.ID.NO.: 46);

SerSerChgGln|SerVal (SEQ.ID.NO.: 47);

SerSerChgGln|SerPro (SEQ.ID.NO.: 48);

SerSerChgGln|SerSer (SEQ.ID.NO.: 49);

SerSerSerChgGln|SerLeu (SEQ.ID.NO.: 50);

SerSerSerChgGln|SerVal (SEQ.ID.NO.: 51);

SerSerSerChgGln|SerPro (SEQ.ID.NO.: 52);

SerSerSerChgGln|SerSer (SEQ.ID.NO.: 53);

SerAlaSerChgGln|SerLeu (SEQ.ID.NO.: 54);

SerAlaSerChgGln|SerVal (SEQ.ID.NO.: 55);

(N-methyl-Ser)SerSerChgGln|SerLeu (SEQ.ID.NO.: 56);

(N-methyl-Ser)SerSerChgGln|SerVal (SEQ.ID.NO.: 57);

4-HypSerSerTyrGln|SerVal (SEQ.ID.NO.: 58);

4-HypSerSerTyrGln|SerLeu (SEQ.ID.NO.: 59);

4-HypSerSerChgGln|SerVal (SEQ.ID.NO.: 60);

4-HypSerSerChgGln|SerLeu (SEQ.ID.NO.: 61);

4-HypSerSerChgGln|SerSer (SEQ.ID.NO.: 62);

4-HypSerSerChgGln|SerSer (SEQ.ID.NO.: 63);

4-HypSerSerChgGln|SerPro (SEQ.ID.NO.: 64);

4-HypSerSerChgGln|SerPro (SEQ.ID.NO.: 65);

4-HypAlaSerChgGln|SerVal (SEQ.ID.NO.: 66);

4-HypAlaSerChgGln|SerLeu (SEQ.ID.NO.: 67);

(3,4-DiHyp)SerSerTyrGln|SerVal (SEQ.ID.NO.: 68); and

(3,4-DiHyp)SerSerTyrGln|SerLeu (SEQ.ID.NO.: 69);

wherein 4-Hyp is 4-hydroxyproline, 3,4-DiHyp is 3,4-dihydroxyproline and Chg is cyclohexylglycine.

The phrase “oligopeptide comprise an amino acid sequence” as used hereinabove, and elsewhere in the Detailed Description of the Invention, describes oligomers of from about 3 to about 100 amino acids residues which include in their amino acid sequence the specific amino acid sequence decribed and which are therefore proteolytically cleaved within the amino acid sequence described by free PSA. Preferably, the oligomer is from 5 to 10 amino acid residues. Thus, for example, the following oligomer:

hArgSerAlaChgGln|SerLeu (SEQ.ID.NO.: 70); comprises the amino acid sequence: ChgGln|SerLeu (SEQ.ID.NO.: 12); and would therefore come within the instant invention. And the oligomer: hArgSer4-HypChgGln|SerLeu (SEQ.ID.NO.: 71); comprises the amino acid sequence: 4-HypChgGln|SerLeu (SEQ.ID.NO.: 72); and would therefore come within the instant invention. It is understood that such oligomers do not include semenogelin I and semenogelin II.

A person of ordinary skill in the peptide chemistry art would readily appreciate that certain amino acids in a biologically active oligopeptide may be replaced by other homologous, isosteric and/or isoelectronic amino acids wherein the biological activity of the original oligopeptide has been conserved in the modified oligopeptide. Certain unnatural and modified natural amino acids may also be utilized to replace the corresponding natural amino acid in the oligopeptides of the instant invention. Thus, for example, tyrosine may be replaced by 3-iodotyrosine, 2-methyltyrosine, 3-fluorotyrosine, 3-methyltyrosine and the like. Further for example, lysine may be replaced with N′-(2-imidazolyl)lysine and the like. The following list of amino acid replacements is meant to be illustrative and is not limiting:

| Original Amino Acid | Replacement Amino Acid(s) |
|---|---|
| Ala | Gly |
| Arg | Lys, Ornithine |
| Asn | Gln |
| Asp | Glu |
| Glu | Asp |
| Gln | Asn |
| Gly | Ala |
| Ile | Val, Leu, Met, Nle |
| Leu | Ile, Val, Met, Nle |
| Lys | Arg, Ornithine |
| Met | Leu, Ile, Nle, Val |
| Ornithine | Lys, Arg |
| Phe | Tyr, Trp |
| Ser | Thr |
| Thr | Ser |
| Trp | Phe, Tyr |
| Tyr | Phe, Trp |
| Val | Leu, Ile, Met, Nle |

Thus, for example, the following oligopeptides may be synthesized by techniques well known to persons of ordinary skill in the art and would be expected to be proteolytically cleaved by free PSA:

AsnArgIleSerTyrGln|Ser (SEQ.ID.NO.: 73)

AsnLysValSerTyrGln|Ser (SEQ.ID.NO.: 74)

AsnLysMetSerTyrGln|SerSer (SEQ.ID.NO.: 75)

AsnLysLeuSerTyrGln|SerSer (SEQ.ID.NO.: 76)

AsnLysIleSerTyrGln|Ser (SEQ.ID.NO.: 77)

GlnLysIleSerTyrGln|SerSer (SEQ.ID.NO.: 78).

Asn4-HypIleSerTyrGln|Ser (SEQ.ID.NO.: 79)

Asn4-HypValSerTyrGln|Ser (SEQ.ID.NO.: 80)

4-HypAlaSerTyrGln|SerSer (SEQ.ID.NO.: 81)

(3,4-dihydroxyproline)AlaSerTyrGln|SerSer (SEQ.ID.NO.: 82)

3-hydroxyprolineSerChgGln|Ser (SEQ.ID.NO.: 83)

4-HypAlaSerChgGln|SerSer (SEQ.ID.NO.: 84).

The inclusion of the symbol “|” within an amino acid sequence indicates the point within that sequence where the oligopeptide is proteolytically cleaved by free PSA.

The compounds of the present invention may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration

In the present invention, the amino acids which are disclosed are identified both by conventional 3 letter and single letter abbreviations as indicated below:

| | | |
|---|---|---|
| Alanine | Ala | A |
| Arginine | Arg | R |
| Asparagine | Asn | N |
| Aspartic acid | Asp | D |
| Asparagine or | Asx | B |
| Aspartic acid | | |
| Cysteine | Cys | C |
| Glutamine | Gln | Q |
| Glutamic acid | Glu | E |
| Glutamine or | | |
| Glutamic acid | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The following abbreviations are utilized in the specification and figures to denote the indicated amino acids and moieties:

| | |
|---|---|
| hR or hArg: | homoarginine |
| hY or hTyr: | homotyrosine |
| Cha: | cyclohexylalanine |
| Amf: | 4-aminomethylphenylalanine |
| DAP: | 1,3-diaminopropyl |
| DPL: | 2-(4,6-dimethylpyrimidinyl)lysine |
| (imidazolyl)K: | N′-(2-imidazolyl)lysine |
| Me₂PO₃—Y: | O-dimethylphosphotyrosine |
| O-Me-Y: | O-methyltyrosine |
| TIC: | 1,2,3,4-tetrahydro-3-isoquinoline carboxylic acid |
| DAP: | 1,3-diaminopropane |
| TFA: | trifluoroacetic acid |
| AA: | acetic acid |
| 3PAL: | 3-pyridylalanine |
| 4-Hyp: | 4-hydroxyproline |
| Trt: | trityl |

It is well known in the art, and understood in the instant invention, that peptidyl therapeutic agents such as the instant oligopeptide-therapeutic agent conjugates preferably have the terminal amino moiety of any oligopeptide substituent protected with a suitable protecting group, such as acetyl, benzoyl, pivaloyl and the like. Such protection of the terminal amino group reduces or eliminates the enzymatic degradation of such peptidyl therapeutic agents by the action of exogenous amino peptidases which are present in the blood plasma of warm blooded animals. Such protecting groups also include hydrophilic blocking groups, which are chosen based upon the presence of hydrophilic functionality. Blocking groups that increase the hydrophilicity of the conjugates and therefore increase the aqueous solubility of the conjugates include but are not limited to hydroylated alkanoyl, polyhydroxylated alkanoyl, polyethylene glycol, glycosylates, sugars and crown ethers. N-Terminus unnatural amino acid moieties may also ameleorate such enzymatic degradation by exogenous amino peptidases.

Preferably the N-terminus protecting group is selected from

a) acetyl;

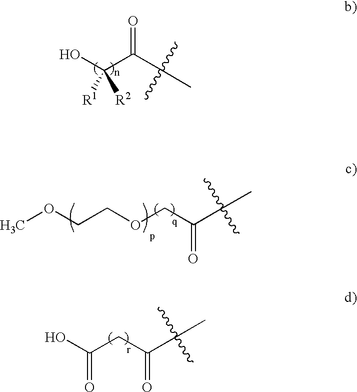

wherein:

R¹ and R² are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R⁶O—, R⁶C(O)NR⁶—, (R⁶)₂NC(O)—, R⁶₂N—C(NR⁶)—, R⁷S(O)₂NH, CN, NO₂, R⁶C(O)—, N₃, —N(R⁶)₂, or R⁷OC(O)NR⁶—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R⁶O—, R⁷S(O)₂NH, R⁶C(O)NR⁶—, (R⁶)₂NC(O)—, R⁶₂N—C(NR⁶)—, CN, R⁶C(O)—, N₃, —N(R⁶)₂, and R⁷OC(O)—NR⁶—; or

R¹ and R² are combined to form —(CH₂)ₛ — wherein one of the carbon atoms is optionally replaced by a moiety selected from: O, S(O)ₘ, —NC(O)—, NH and —N(COR⁷)—;

R⁶ is selected from: hydrogen, aryl, substituted aryl, heterocycle, substituted heterocycle, C₁-C₆ alkyl and C₃-C₁₀ cycloalkyl;

R⁷ is selected from: aryl, substituted aryl, heterocycle, substituted heterocycle, C₁-C₆ alkyl and C₃-C₁₀ cycloalkyl;

m is 0, 1 or 2;

n is 1, 2, 3 or 4;

p is zero or an integer between 1 and 100; and

q is 0 or 1, provided that if p is zero, q is 1; and

r is 1, 2 or 3;

s is 3, 4 or 5.

The instant conjugate also comprises a peptide or small molecule that binds to an anti-apoptotic Bcl-2 family protein, inhibits the expression of such a Bcl-2 family protein or inhibits the function of such a Bcl-2 family protein in another way (termed in general “therapeutic agents”). Examples of small molecules that bind to Bcl-2 family proteins have been described in PCT Publ. Nos. WO 00/04901 and WO 01/14365, J. -L. Wang et al. PNAS 97:7124-7129 (2000) and Nature Cell Biology, 3:173-182 (2001). Examples of peptides that bind to a Bcl-2 family protein have been described in PCT Publ. Nos. WO 98/58541 and WO 00/59526 and J. -L. Wang et al. Cancer Res. 60:1498-1502 (2000).

Examples of oligonucleotides that inhibit the expression of a Bcl-2 family protein are described in U.S. Pat. No. 6,001,992 and in PCT Publ. Nos. WO 00/01393, WO 00/20432 and WO 00/66724.

The preferred peptides or small molecules that bind to an anti-apoptotic Bcl-2 family protein include compounds of the following formulae:

i) ANTIMYCINS OF FORMULA (I):

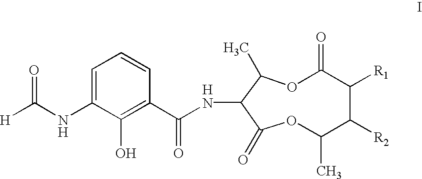

in which

R₁ is H or C₁-C₁₀ alkyl

R₂ is hydrogen, —OH, or —(C₁-C₁₀ alkyl)-CO₂H;

or a pharmaceutically acceptable salt or optical isomer thereof;

ii) compounds of the formula II as described in PCT Publ. No. WO 00/04901:

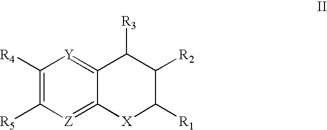

wherein:

X is selected from the group consisting of CH₂; CHOCH₃; NH; O; and S;

Y and Z are independently selected from the group consisting of CH and N; and when Z is N, then Y may further be —CR₆, where R₆ is selected from the group consisting of CH₃ ; OCH₃; CNH₂; and COH;

R₁ is selected from the group consisting of hydrogen; C₁₋₅alkyl; C₁₋₅alkoxy; OH; NH₂; NO₂; CHO; COCH₃; COOH; COOCH₃; N(C₁₋₃alkyl)₂; NH(C₁₋₃alkyl); OCOCH₃; OCOCH₂CH₃; NHCOCH₃; NHNHCOCH₃; NHNHCONH₂; phenyl; phenyl which is mono-, di-, or tri-substituted with NH₂, OH, halogen, NO₂, CF₃, COOH or COOCH₃; cyclohexyl; cyclohexyl which is mono- di-, or tri-substituted with NH₂, OH, halogen or CF₃; and five- and six-member heterocyclic rings, preferably a heterocyclic ring selected from the group consisting of piperidino, piperazino, morpholino, pyrimidyl, pyrrolidino and imidazo;

R₂ is selected from the group consisting of hydrogen; C₁₋₃alkyl C₁₋₃alkoxy; halogen; CF₃; NH₂; OH; COOH; COOCH₃; CONH₂; and CONHCH₃;

or R₁ and R₂ together may form the group —CH₂CH₂CH₂— or —CH₂CH₂CH₂CH₂—;

or R₁ and R₂ together may form, starting from R₁, the group —NHCH₂CH₂, —NHCOCH₂—, or —OCOCH₂—;

R₃ is selected from the group consisting of H; CH₃; CF₃; OCH₃; NH₂; OH; COOH; COCH₃; CH═CH₂; CH═CHCH₂; CH(CH₃)₂; CH₂OH; CH₂NH₂; CH₂COOH; cyclohexyl; cyclohexyl which is mono- di-, or tri-substituted with NH₂, OH, halogen, OCH₃ or CF₃; five- and six-member heterocyclic rings, preferably a heterocyclic ring selected from the group consisting of piperidinyl, piperazinyl, morpholino, pyrimidyl, pyrrolyl, pyrrolidino, and imidazyl; and a substituted phenyl group of the formula:

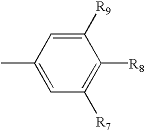

wherein

R₇, R₈ and R₉ are independently selected from the group consisting of hydrogen, CH₃, CF₃, OH, OCH₃, CH₂OH and CHO; provided that at least two of the members of the group R₇, R₈ and R₉ must be OH or OCH₃ when the remaining member of the group is hydrogen, CH₃ or CF₃;

R₄ and R₅ are independently selected from the group consisting of hydrogen, CH₃, and OCH₃; and when Y and Z are both CH, R₄ and R₅ may be further selected from OH and NH₂;

or, R₄ and R₅ together may form the group —CH₂CH₂CH₂— or —CH₂CH₂CH₂CH₂—;

or, R₄ and R₅ together may form, starting from R₄, the group —NHCH₂CH₂—, —NHCOCH₂—, OCOCH₂— or —O(CH₂)nO—, wherein n is 1, 2 or 3;

or a pharmaceutically acceptable salt thereof when the compound includes at least on NH₂ or COOH substituent;

iii) a compound of the formula III as described in PCT Publ. No. WO 00/04901:

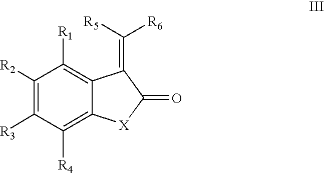

wherein

R₁, R₂, R₃ and R₄ are independently selected from the group consisting of hydrogen; C₁₋₅alkyl; C₁₋₅alkoxy; OH; NH₂; NO₂; CHO; COCH₃; COOH; COOCH₃; N(C₁₋₃alkyl)₂; and NH(C₁₋₃alkyl); and one of R₁, R₂, R₃ and R₄ may be phenyl or a heterocyclic ring, preferably a heterocyclic ring selected from the group consisting of piperidino, piperazino, morpholino, pyrimidyl, pyrrolidino and imidazo; provided at least one of R₁, R₂, R₃ and R₄ must be hydrogen;

R₅ and R₆ are independently selected from the group consisting of hydrogen; CN; CH₂CN; COOCH₃; CONH₂; phenyl; phenyl which is mono-, di-, or tri-substituted with NH₂, OH, halogen, NO₂, CH₃, OCH₃, CF₃, COOH or COOCH₃; cyclohexyl; cyclohexyl which is mono-, di-, or tri-substituted with NH₂, OH, halogen or CF₃; and five- and six-member heterocyclic rings, preferably a heterocyclic ring selected from the group consisting of pyrrolyl, imidazolyl, piperidinyl, piperazinyl, morpholino, pyrimidyl and pyrrolidino; provided, only one of R₅ or R₆ may be phenyl, substituted phenyl, cyclohexyl, substituted cyclohexyl or heterocyclic in the same compound, and further provided that when one of R₅ or R₆ is phenyl, substituted phenyl, cyclohexyl, substituted cyclohexyl or heterocyclic, then the other must be hydrogen;

or at least one of R₅ and R₆ may be halogen, provided that the other must be C₁₋₅alkyl or C₁₋₅alkoxy;

or a pharmaceutically acceptable salt thereof when the compound included at least one NH₂ or COOH substituent;

iv) a compound of the formula IV as described in PCT Publ. No. WO 00/04901:

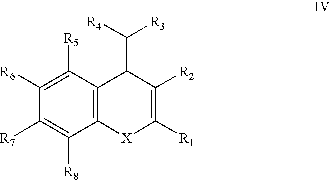

wherein:

X is selected from the group consisting of CH₂; CHOCH₃; NH; NCH₃; O; and S;

R₁ is selected from the group consisting of OH; NH₂; CHO; COCH₃; COOH; N(C₁₋₃alkyl); OCOCH₃; OCOCH₂CH₃; NHCOCH₃; NHNHCOCH₃; NHNHCONH₂; N(C₁₋₃alkyl)₂; NH(C₁₋₃alkyl); and five- and six-member heterocyclic rings, preferably a heterocyclic ring selected form the group consisting piperidinyl, piperazinyl, morpholino, pyrimidyl, pyrrolyl, pyrrolidino and imidazyl;

R₂ is selected from the group consisting of C₁₋₃alkyl; C₁₋₃alkoxy; OH: NH₂; CHO; COCH₃; OCOCH₃; OCOCH₂CH₃; COOH; COOCH₃; COOCH₂CH₃; COOCH₂CH₂CH₃;

R₃ is selected from the group consisting of C₁₋₃alkyl; C₁₋₃alkoxy; CN; CH₂CN; CH₂NO₂; CHO; COCH₃; COOH; OCOCH₃; OCOCH₂CH₃; NHCOCH₃; NHNHCOCH₃; NHNHCONH₂; CH═CH₂; CH₂CH═CH₂; CH₂CHO; and five- and six-member heterocyclic rings, preferably a heterocyclic ring selected from the group consisting piperidinyl, piperazinyl, morpholino, pyrimidyl, pyrrolyl, pyrrolidino and imidazyl;

R₄ is selected from the group consisting of C₁₋₃alkyl C₁₋₃alkoxy; CN; CH₂CN; CH₂NO₂; CHO; COCH₃; COCH₃; COOH; COOCH₃; COOCH₂CH₃; COOCH₂CH₂CH₃; OCOCH₃; OCOCH₂, CH₃;

R₅ is selected from the group consisting of hydrogen CH₃; OCH₃; OH; NH₂; Br; Cl; and F; and

R₆, R₇ and R₈ are selected from the group consisting of hydrogen, CH₃; CH₂CH₃; CF₃; NH₂; OH; OCH₃; CN; NO₂; CL; Br; F; COOH; and COOCH₃; provided, at least one member of the group R₆, R₇ or R₈ must be Cl, Br or F when the remaining members of said group are hydrogen;

or a pharmaceutically acceptable salt thereof when the compound includes at least one NH₂ or COOH substituent;

v) a compound of the formula V

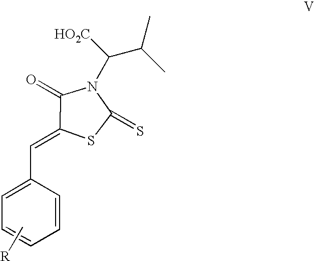

wherein R is selected from H, halogen, NH₂, NH(C₁-C₆alkyl) and N(C₁-C₆alkyl)₂; or a pharmaceutically acceptable salt or optical isomer thereof; and

vi) a compounds of the formula VI:

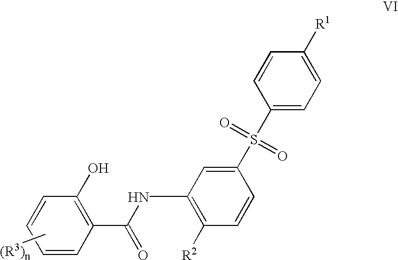

wherein:

R¹ is halogen;

R² is halogen;

R³ is halogen; and

n is 0, 1 or 2

or a pharmaceutically acceptable salt or optical isomer thereof.

Specific examples of compounds of the formula I that are useful as components of the conjugate of the instant invention include:

| | |
|---|---|
| | I |
| 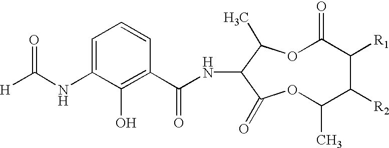 | |

| Name | R₁ | R₂ |
|---|---|---|
| actimycin A₀₍ₐ₎ | hexyl | hexanoic acid |
| actimycin A_{0(b)} | butyl | heptanoic acid |
| antimycin A_{0(c)} | octyl | pentanoic acid |
| antimycin A_{0(d)} | heptyl | pentanoic acid |
| antimycin A₁ | hexyl | isovaleric acid |
| antimycin A₂ | hexyl | butanoic acid |
| antimycin A₃ | butyl | isobutanoic acid |
| antimycin A₄ | butyl | butanoic acid |
| antimycin A₅ | ethyl | isobutanoic acid |
| antimycin A₆ | ethyl | butanoic acid |
| kitamycin A | hexyl | hydroxyl |
| kitamycin B | isohexyl | hydroxyl |
| urauchimycin B | isohexyl | hydroxyl |
| deisovalerylblastomycin | butyl | hydroxyl |
| dehexyl-deisovalerylblastomycin | hydrogen | hydrogen |

Specific example of compounds of the formula II that are useful as components of the conjugate of the instant invention include:

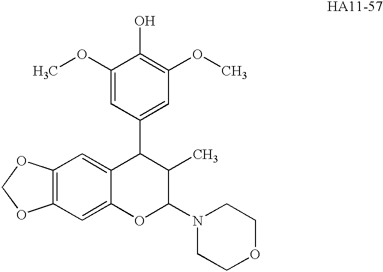

Specific examples of compounds of the formula IV that are useful as components of the conjugate of the instant invention include:

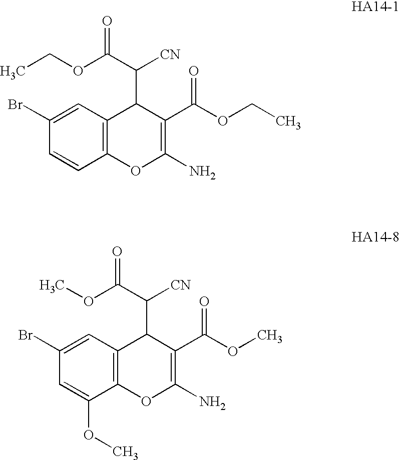

Specific examples of compounds of the formula V that are useful as components of the conjugate of the instant invention include:

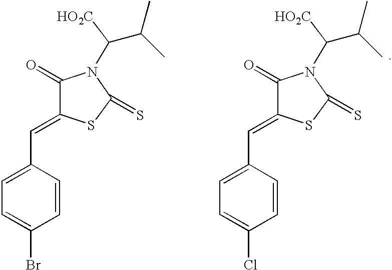

Specific examples of compounds of the formula VI that are useful as components of the conjugate of the instant invention include:

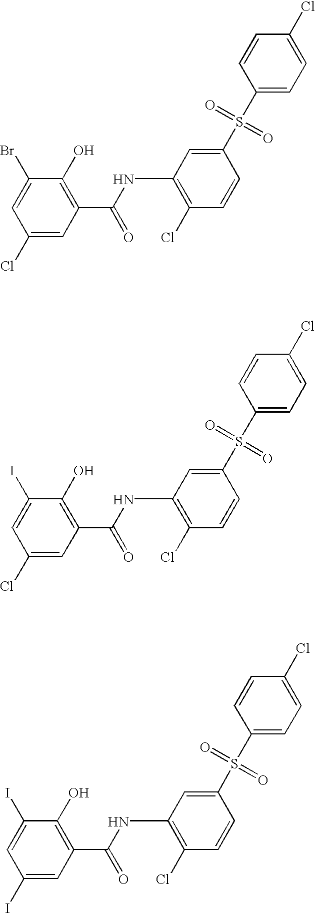

The conjugate of the instant invention wherein the therapeutic agent is a compound of the formula I may be described by the general formula A below:

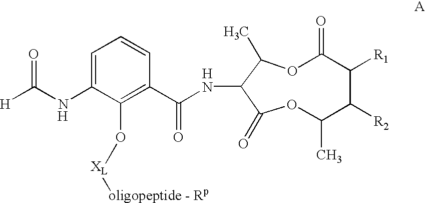

wherein:

oligopeptide is an oligopeptide which is selectively recognized by enzymatically active prostate specific antigen (PSA) and is capable of being proteolytically cleaved by the enzymatic activity of the prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R₁ is H or C₁-C₁₀ alkyl;

R₂ is hydrogen, —OH, or —(C₁-C₁₀ alkyl)-CO₂H;

R^{p} is selected from

a) hydrogen,

b) —(C═O)R^{a},

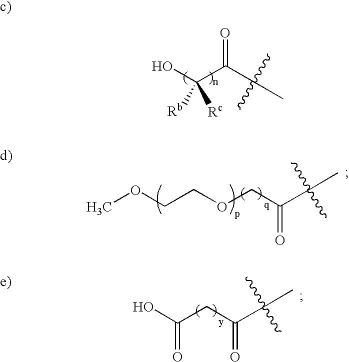

f) ethoxysquarate; and

g) cotininyl;

R^{b} and R^{c} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or

R^{b} and R^{c} are combined to form —(CH₂)ₛ— wherein one of the carbon atoms is optionally replaced by a moiety selected from: O, S(O)ₘ, —NC(O)—, NH and —N(COR^{e})—;

R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,

R^{d} is selected from: hydrogen, aryl, substituted aryl, heterocycle, substituted heterocycle, C₁-C₆ alkyl and C₃-C₁₀ cycloalkyl;

R^{e} is selected from: aryl, substituted aryl, heterocycle, substituted heterocycle, C₁-C₆ alkyl and C₃-C₁₀ cycloalkyl;

W is selected from a bond, a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

u is selected from: 0, 1, 2 or 3;

y is 1, 2 or 3;

or the pharmaceutically acceptable salt thereof.

Preferably, u is 1.

Preferably, R^{b} and R^{c} are independently selected from: hydrogen, OH, C₁-C₆ alkyl, C₁-C₆ alkoxy, C₁-C₆ aralkyl and aryl.

The conjugate of the instant invention wherein the therapeutic agent is a compound of the formula II may be described by the general formula B below:

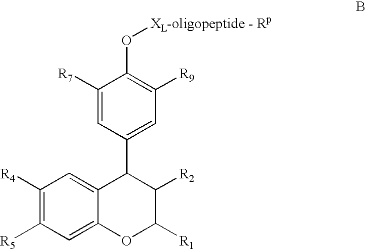

wherein:

oligopeptide is an oligopeptide which is selectively recognized by enzymatically active prostate specific antigen (PSA) and is capable of being proteolytically cleaved by the enzymatic activity of the prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R₁ is selected from the group consisting of hydrogen; C₁₋₅ alkyl; C₁₋₅ alkoxy; OH; NH₂; NO₂; CHO; COCH₃; COOH; COOCH₃; N(C₁₋₃alkyl)₂; NH(C₁₋₃alkyl); OCOCH₃; OCOCH₂CH₃; NHCOCH₃; NHNHCOCH₃; NHNHCONH₂; phenyl; phenyl which is mono-, di-, or tri-substituted with NH₂, OH, halogen, NO₂, CF₃, COOH or COOCH₃; cyclohexyl; cyclohexyl which is mono- di-, or tri-substituted with NH₂, OH, halogen or CF₃; and five- and six-member heterocyclic rings, preferably a heterocyclic ring selected from the group consisting of piperidino, piperazino, morpholino, pyrimidyl, pyrrolidino and imidazo;

R₂ is selected from the group consisting of hydrogen; C₁₋₃alkyl C₁₋₃alkoxy; halogen; CF₃; NH₂; OH; COOH; COOCH₃; CONH₂; and CONHCH₃;

or R₁ and R₂ together may form the group —CH₂CH₂CH₂— or —CH₂CH₂CH₂CH₂—;

or R₁ and R₂ together may form, starting from R₁, the group —NHCH₂CH₂, —NHCOCH₂—, or —OCOCH₂—;

R₇ and R₉ are independently selected from the group consisting of hydrogen, CH₃, CF₃, OH, OCH₃, CH₂OH and CHO; provided that at least of R₇ and R₉ must be OH or OCH₃ when the remaining member of the group is hydrogen, CH₃ or CF₃;

R^{p} is selected from

a) hydrogen,

b) —(C═O)R^{a},

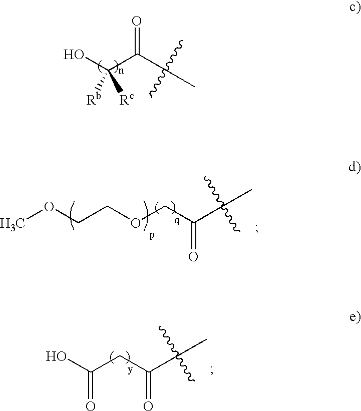

f) ethoxysquarate; and

g) cotininyl;

R^{b} and R^{c} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or

R^{b} and R^{c} are combined to form —(CH₂)ₛ— wherein one of the carbon atoms is optionally replaced by a moiety selected from: O, S(O)ₘ, —NC(O)—, NH and —N(COR^{e})—;

R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,

R^{d} is selected from: hydrogen, aryl, substituted aryl, heterocycle, substituted heterocycle, C₁-C₆ alkyl and C₃-C₁₀ cycloalkyl;

R^{e} is selected from: aryl, substituted aryl, heterocycle, substituted heterocycle, C₁-C₆ alkyl and C₃-C₁₀ cycloalkyl;

W is selected from a bond, a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

u is selected from: 0, 1, 2 or 3;

y is 1, 2 or 3;

or the pharmaceutically acceptable salt thereof.

The conjugate of the instant invention wherein the therapeutic agent is a compound of the formula IV may be described by the general formula C below:

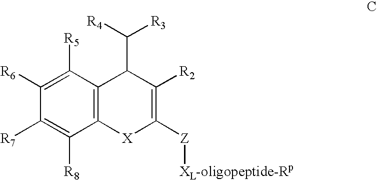

wherein:

oligopeptide is an oligopeptide which is selectively recognized by enzymatically active prostate specific antigen (PSA) and is capable of being proteolytically cleaved by the enzymatic activity of the prostate specific antigen,

X is selected from the group consisting of CH₂, CHOCH₃, NH, NCH₃, O and S;

X_{L} is selected from: a bond, —C(O)—(CH₂)ᵤ—W—(CH₂)ᵤ—O— and —C(O)—(CH₂)ᵤ—W—(CH₂)ᵤ—NH—;

Z is selected from: —O— and —NH—;

R₂ is selected from the group consisting of C₁₋₃alkyl; C₁₋₃alkoxy; OH: NH₂; CHO; COCH₃; OCOCH₃; OCOCH₂CH₃; COOH; COOCH₃; COOCH₂CH₃; COOCH₂CH₂CH₃;

R₃ is selected from the group consisting of C₁₋₃alkyl; C₁₋₃alkoxy; CN; CH₂CN; CH₂NO₂; CHO; COCH₃; COOH; OCOCH₃; OCOCH₂CH₃; NHCOCH₃; NHNHCOCH₃; NHNHCONH₂; CH═CH₂; CH₂CH═CH₂; CH₂CHO; and five- and six-member heterocyclic rings, preferably a heterocyclic ring selected from the group consisting piperidinyl, piperazinyl, morpholino, pyrimidyl, pyrrolyl, pyrrolidino and imidazyl;

R₄ is selected from the group consisting of C₁₋₃alkyl C₁₋₃alkoxy; CN; CH₂CN; CN₂NO₂; CHO; COCH₃; COCH₃; COOH; COOCH₃; COOCH₂CH₃; COOCH₂CH₂CH₃; OCOCH₃; OCOCH₂, CH₃;

R₅ is selected from the group consisting of hydrogen CH₃; OCH₃; OH; NH₂; Br; Cl; and F; and

R₆, R₇ and R₈ are selected from the group consisting of hydrogen, CH₃; CH₂CH₃; CF₃; NH₂; OH; OCH₃; CN; NO₂; CL; Br; F; COOH; and COOCH₃; provided, at least one member of the group R₆, R₇ or R₈ must be Cl, Br or F when the remaining members of said group are hydrogen;

R^{p} is selected from

a) hydrogen,

b) —(C═O)R^{a},

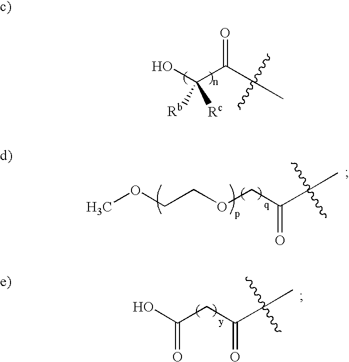

f) ethoxysquarate; and

g) cotininyl;

R^{b} and R^{c} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or

R^{b} and R^{c} are combined to form —(CH₂)ₛ— wherein one of the carbon atoms is optionally replaced by a moiety selected from: O, S(O)ₘ, —NC(O)—, NH and —N(COR^{e})—;

R^{a} is C₁-C₆-alkyl, hydroxylated C₃-C₈-cycloalkyl, polyhydroxylated C₃-C₈-cycloalkyl, cycloalkyl, hydroxylated aryl, polyhydroxylated aryl or aryl,

R^{d} is selected from: hydrogen, aryl, substituted aryl, heterocycle, substituted heterocycle, C₁-C₆ alkyl and C₃-C₁₀ cycloalkyl;

R^{e} is selected from: aryl, substituted aryl, heterocycle, substituted heterocycle, C₁-C₆ alkyl and C₃-C₁₀ cycloalkyl;

W is selected from a bond, a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

u is selected from: 0, 1, 2 or 3;

y is 1, 2 or 3;

or the pharmaceutically acceptable salt thereof.

The conjugate of the instant invention wherein the therapeutic agent is a compound of the formula V may be described by the general formula D below:

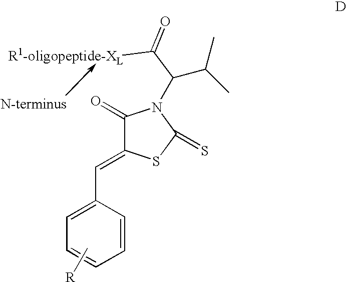

wherein:

oligopeptide is an oligopeptide which is selectively recognized by enzymatically active prostate specific antigen (PSA) and is capable of being proteolytically cleaved by the enzymatic activity of the prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R is selected from H, halogen, NH₂, NH(C₁-C₆ alkyl) and N(C₁-C₆ alkyl)₂; and

R¹ is selected from H, C₁-C₁₈ alkyl, benzyl, aryl, NH₂, NH(C₁-C₆ alkyl), N(C₁-C₆ alkyl)₂, morpholinyl and piperidinyl;

W is selected from a bond, a branched or straight chain C₁-C₆-alkyl, cyclopentyl, cyclohexyl, cycloheptyl or bicyclo[2.2.2]octanyl;

u is selected from: 0, 1, 2 or 3;

or a pharmaceutically acceptable salt or optical isomer thereof thereof.

The conjugate of the instant invention wherein the therapeutic agent is a compound of the formula VI may be described by the general formula E below:

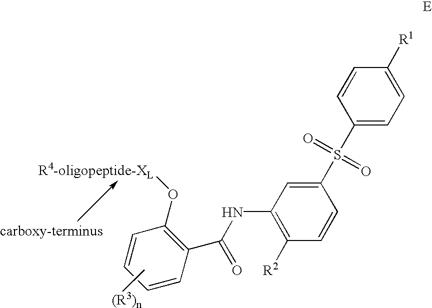

wherein:

oligopeptide is an oligopeptide which is selectively recognized by enzymatically active prostate specific antigen (PSA) and is capable of being proteolytically cleaved by the enzymatic activity of the prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R¹ is halogen;

R² is halogen;

R³ is halogen; and

n is 0, 1 or 2;

R⁴ is selected from

a) hydrogen,

b) —(C═O)R^{a},

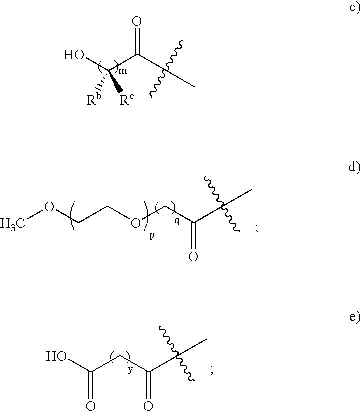

f) ethoxysquarate; and

g) cotininyl;

R^{b} and R^{c} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or

R^{b} and R^{c} are combined to form —(CH₂)ₛ— wherein one of the carbon atoms is optionally replaced by a moiety selected from: O, S(O)ₘ, —NC(O)—, NH and —N(COR^{e})—;

R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,

R^{d} is selected from: hydrogen, aryl, substituted aryl, heterocycle, substituted heterocycle, C₁-C₆ alkyl and C₃-C₁₀ cycloalkyl;

R^{e} is selected from: aryl, substituted aryl, heterocycle, substituted heterocycle, C₁-C₆ alkyl and C₃-C₁₀ cycloalkyl;

W is selected from a bond, a branched or straight chain C₁-C₆-alkyl, cyclopentyl, cyclohexyl, cycloheptyl or bicyclo[2.2.2]octanyl;

m is 1, 2, 3 or 4;

p is zero or an integer between 1 and 100;

q is 0 or 1, provided that if p is zero, q is 1;

u is selected from: 0, 1, 2 or 3;

y is 1, 2 or 3;

or a pharmaceutically acceptable salt or optical isomer thereof

Examples of compounds of the instant invention include:

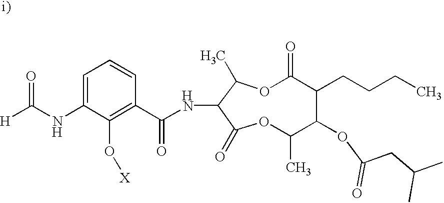

wherein X is

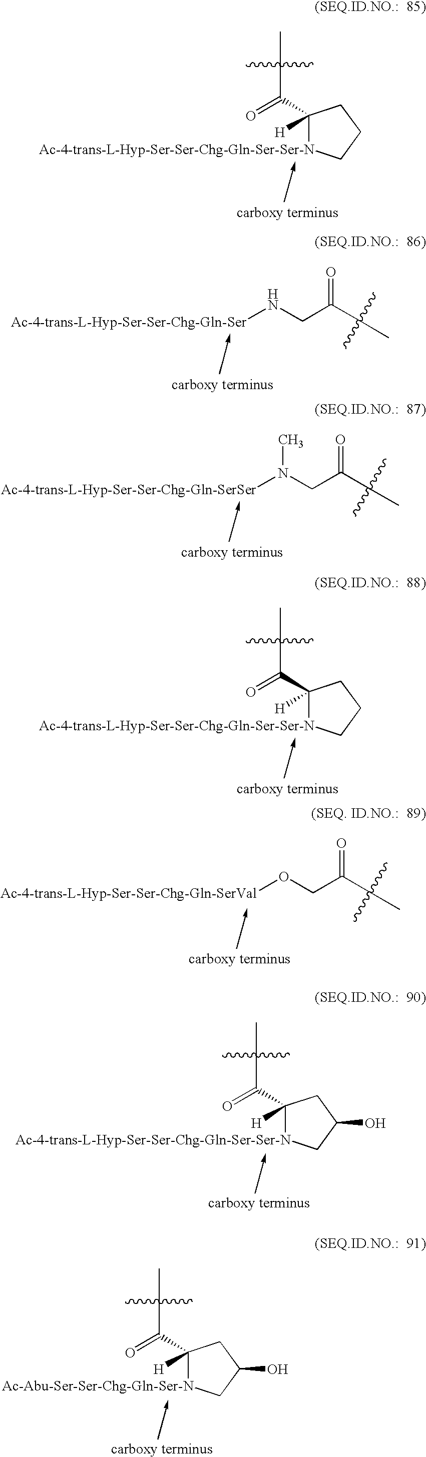

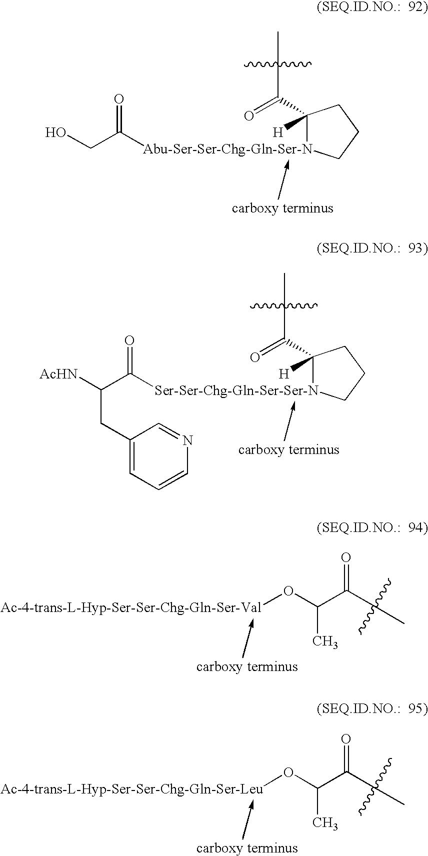

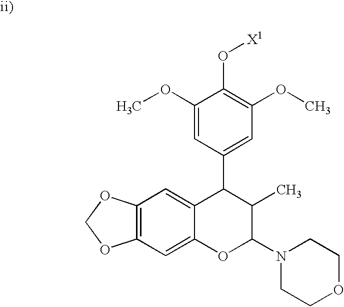

wherein X¹ is

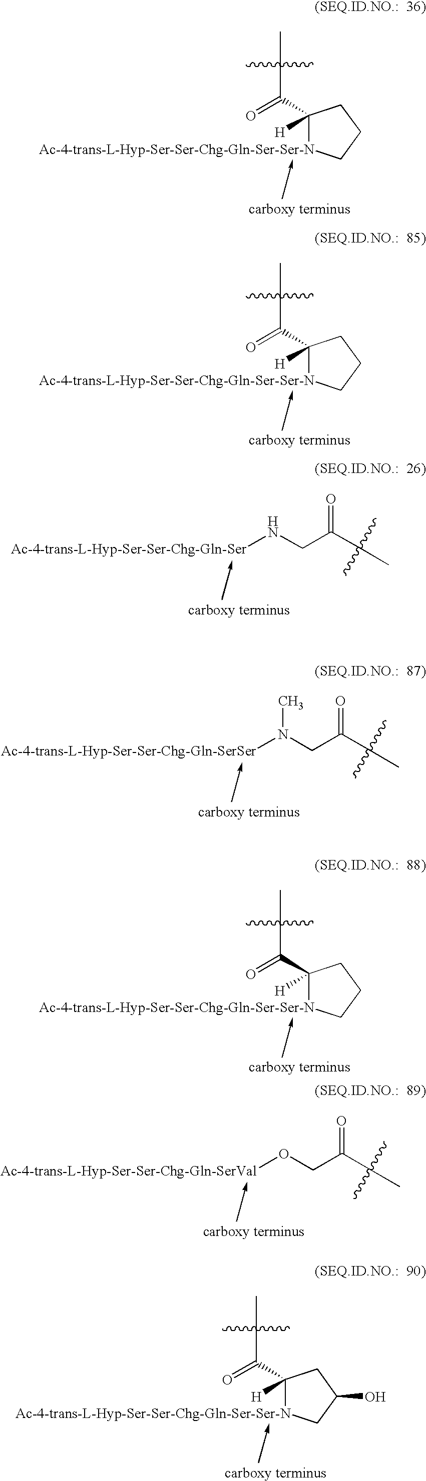

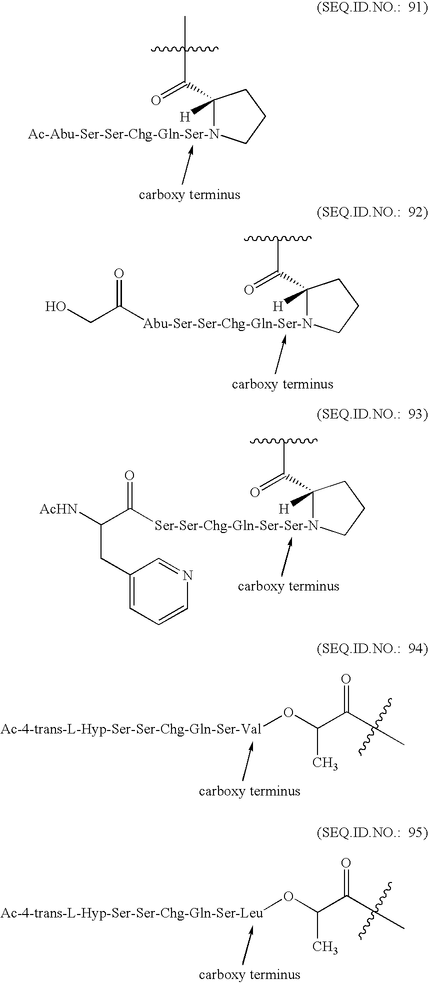

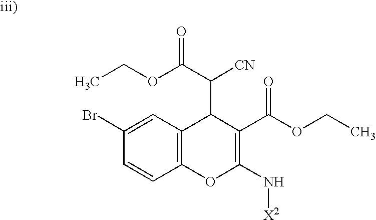

wherein X² is

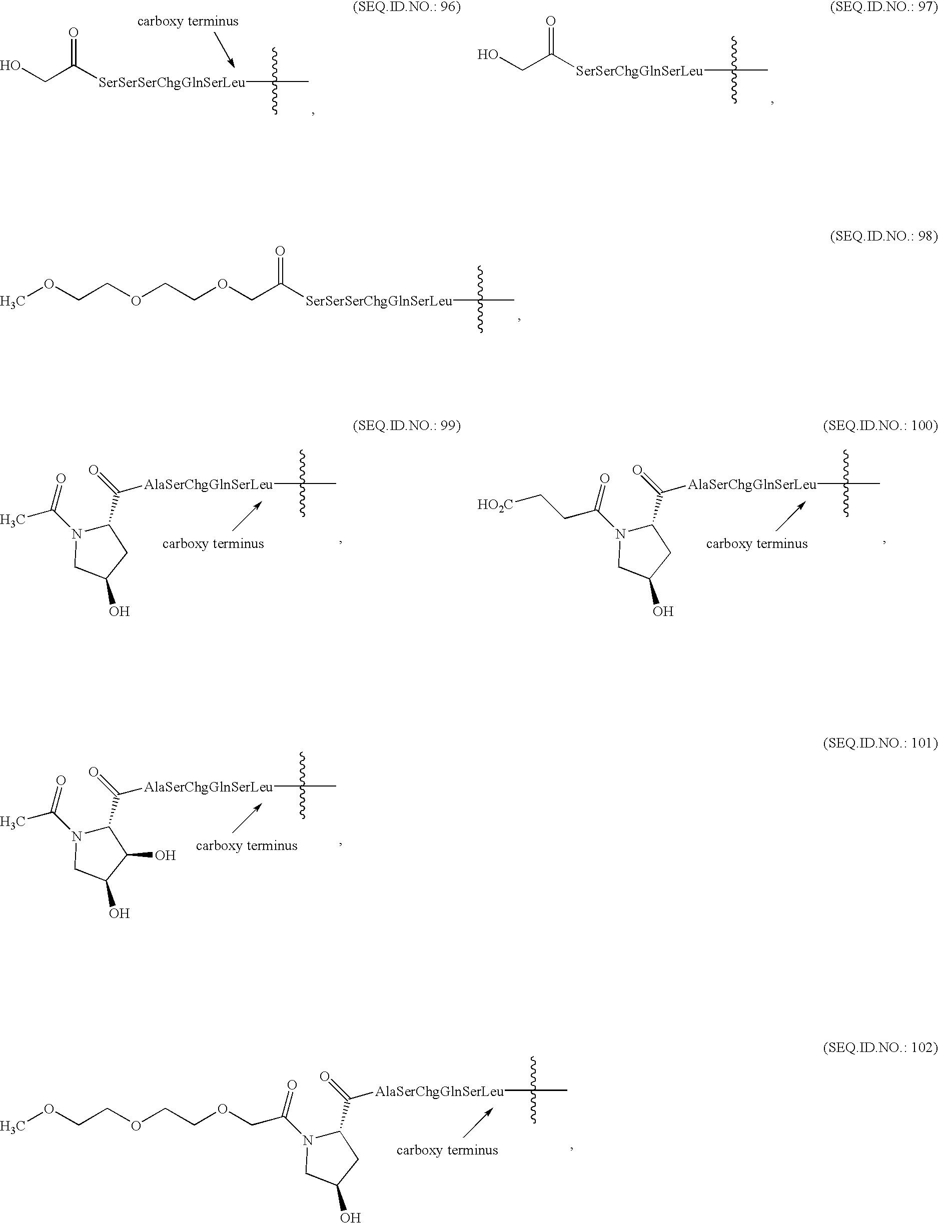

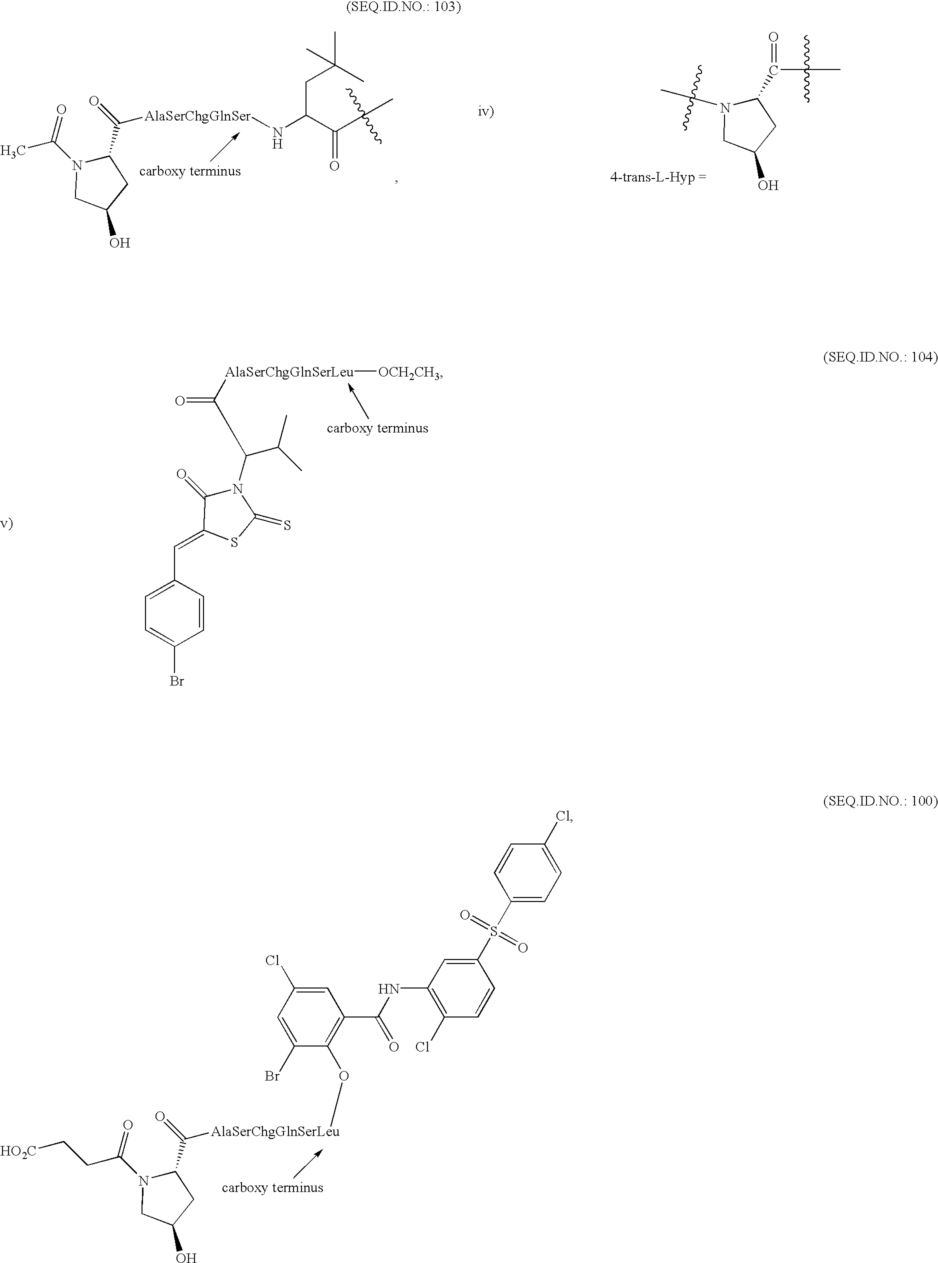

Certain of the oligopeptides of the instant conjugates comprise a cyclic amino acid substituted with a hydrophilic moiety, previously represented by the term “Haa”, which may also be represented by the formula:

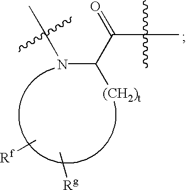

wherein:

R^{f} is selected from HO— and C₁-C₆ alkoxy;

R^{g} is selected from hydrogen, halogen, C₁-C₆ alkyl, HO— and C₁-C₆ alkoxy; and

t is 3 or 4.

The structure

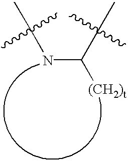

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

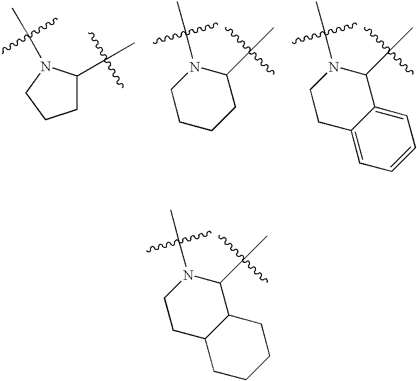

The conjugates of the present invention may have asymmetric centers and occur as racemates, racemic mixtures, and as individual diastereomers, with all possible isomers, including optical isomers, being included in the present invention. When any variable (e.g. aryl, heterocycle etc.) occurs more than one time in any constituent, its definition on each occurrence is independent of every other occurence. Also, combinations of substituents and/or variables are permissible only if such combinations result in stable compounds.

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “chlorosubstituted-alkyl” is intended to include both branched and straight-chain saturated aliphatic hydrocarbon groups having the specified number of carbon atoms and being substituted with a chlorine atom. Examples include, but are not limited to chloromethyl, 1-chloroethyl, 2-chloroethyl, 1-chloropropyl, 2-chloropropyl and the like.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Alkenyl” groups include those groups having the specified number of carbon atoms and having one or several double bonds. Examples of alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, geranylgeranyl and the like.

“Alkynyl” groups include those groups having the specified number of carbon atoms and having one triple bonds. Examples of alkynyl groups include acetylene, 2-butynyl, 2-pentynyl, 3-pentynyl and the like.

“Halogen” or “halo” as used herein means fluoro, chloro, bromo and iodo.

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydronaphthyl, indanyl or biphenyl.

The term heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl.

Preferably, heterocycle is selected from benzimidazolyl, 2-imidazolyl, indolyl, isoquinolinyl, morpholinyl, piperidyl, piperazinyl, pyridyl, pyrrolidinyl, 2-piperidinonyl, 2-pyrollidinonyl, quinolinyl, tetrahydrofuryl, tetrahydroisoquinolinyl, and thienyl.

As used herein in the terms “substituted C₁₋₈ alkyl”, “substituted aryl” and “substituted heterocycle” include moieties containing from 1 to 3 substituents in addition to the point of attachment to the rest of the compound. Such additional substituents are selected from F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, N₃, (C₁-C₆ alkyl)OC(O)NH— and C₁-C₂₀ alkyl.

When R^{b} and R^{c} are combined to form —(CH₂)ₛ— or —(CH₂)_{w}—, the cyclic moieties so defined include, but are not limited to:

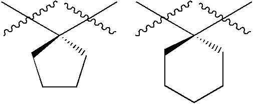

When R^{b} and R^{c} are combined to form —(CH₂)ₛ—, the heteroatom-containing cyclic moieties so defined include, but are not limited to:

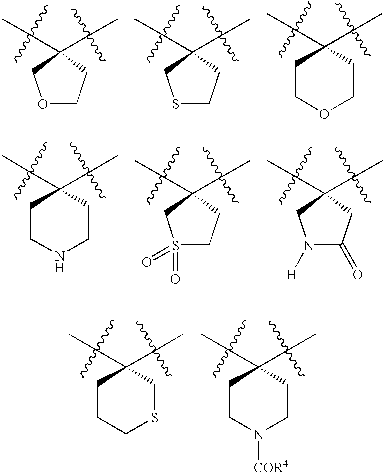

As used herein, the term “hydroxylated” represents substitution on a substitutable carbon of the ring system being so described by a hydroxyl moiety. As used herein, the term “poly-hydroxylated” represents substitution on two or more substitutable carbon of the ring system being so described by two, three or four hydroxyl moieties.

As used herein, the term “cotininyl” represents the following structure:

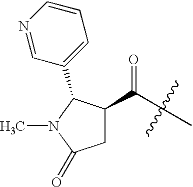

or the diastereomer thereof.

As used herein, the term “4-ethoxysquarate” represents the following structure:

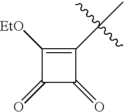

or the pharmaceutically acceptable salt thereof.

The oligopeptides, peptide subunits and peptide derivatives (also termed “peptides”) of the present invention can be synthesized from their constituent amino acids by conventional peptide synthesis techniques, preferably by solid-phase technology. The peptides are then purified by reverse-phase high performance liquid chromatography (HPLC).

Standard methods of peptide synthesis are disclosed, for example, in the following works: Schroeder et al., “The Peptides”, Vol. I, Academic Press 1965; Bodansky et al., “Peptide Synthesis”, Interscience Publishers, 1966; McOmie (ed.) “Protective Groups in Organic Chemistry”, Plenum Press, 1973; Barany et al., “The Peptides: Analysis, Synthesis, Biology” 2, Chapter 1, Academic Press, 1980, and Stewart et al., “Solid Phase Peptide Synthesis”, Second Edition, Pierce Chemical Company, 1984. The teachings of these works are hereby incorporated by reference.

The suitably substituted cyclic amino acid having a hydrophilic substituent, which may be incorporated into the instant conjugates by standard peptide synthesis techniques, is itself either commercially available or is readily synthesized by techniques well known in the art or described herein. Thus syntheses of suitably substituted prolines are described in the following articles and references cited therein: J. Ezquerra et al., J. Org. Chem. 60:2925-2930 (1995); P. Gill and W. D. Lubell, J. Org. Chem., 60:2658-2659 (1995); and M. W. Holladay et al., J. Med. Chem., 34:457-461 (1991). The teachings of these works are hereby incorporated by reference.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The oligopeptide-therapeutic agent conjugates of the invention are useful in the treatment of diseases that are characterized by abnormal cells or abnormal proliferation of cells, whether malignant or benign, wherein those cells are characterized by their secretion of enzymatically active PSA. Such diseases include, but are not limited to, prostate cancer, benign prostatic hyperplasia, metastatic prostate cancer, breast cancer and the like.

The oligopeptide-therapeutic agent conjugates of the invention are administered to the patient in the form of a pharmaceutical composition which comprises a conjugate of of the instant invention and a pharmaceutically acceptable carrier, excipient or diluent therefor. As used, “pharmaceutically acceptable” refers to those agents which are useful in the treatment or diagnosis of a warm-blooded animal including, for example, a human, equine, procine, bovine, murine, canine, feline, or other mammal, as well as an avian or other warm-blooded animal. The preferred mode of administration is parenterally, particularly by the intravenous, intramuscular, subcutaneous, intraperitoneal, or intralymphatic route. Such formulations can be prepared using carriers, diluents or excipients familiar to one skilled in the art. In this regard, See, e.g. Remington's Pharmaceutical Sciences, 16th ed., 1980, Mack Publishing Company, edited by Osol et al. Such compositions may include proteins, such as serum proteins, for example, human serum albumin, buffers or buffering substances such as phosphates, other salts, or electrolytes, and the like. Suitable diluents may include, for example, sterile water, isotonic saline, dilute aqueous dextrose, a polyhydric alcohol or mixtures of such alcohols, for example, glycerin, propylene glycol, polyethylene glycol and the like. The compositions may contain preservatives such as phenethyl alcohol, methyl and propyl parabens, thimerosal, and the like. If desired, the composition can include about 0.05 to about 0.20 percent by weight of an antioxidant such as sodium metabisulfite or sodium bisulfite.

As used herein, the term “composition” is intended to encompass a product comprising the specified ingredients in the specific amounts, as well as any product which results, directly or indirectly, from combination of the specific ingredients in the specified amounts.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For intravenous administration, the composition preferably will be prepared so that the amount administered to the patient will be from about 0.01 to about 1 g of the conjugate. Preferably, the amount administered will be in the range of about 0.2 g to about 1 g of the conjugate. The conjugates of the invention are effective over a wide dosage range depending on factors such as the disease state to be treated or the biological effect to be modified, the manner in which the conjugate is administered, the age, weight and condition of the patient as well as other factors to be determined by the treating physician. Thus, the amount administered to any given patient must be determined on an individual basis.

The conjugates of the instant invention which comprise the oligopeptide containing the PSA cleavage site and a therapeutic agent may similarly be synthesized by techniques well known in the medicinal chemistry art. For example, a free amine moiety on the therapeutic agent may be covalently attached to the oligopeptide at the carboxyl terminus such that an amide bond is formed. Similarly, an amide bond may be formed by covalently coupling an amine moiety of the oligopeptide and a carboxyl moiety of the therapeutic agent. For these purposes a reagent such as 2-(1H-benzotriazol-1-yl)-1,3,3-tetramethyluronium hexafluorophosphate (known as HBTU) and 1-hyroxybenzotriazole hydrate (known as HOBT), dicyclohexylcarbodiimide (DCC), N-ethyl-N-(3-dimethylaminopropyl)-carbodiimide (EDC), diphenylphosphorylazide (DPPA), benzotriazol-1-yl-oxy-tris-(dimethylamino)phosphonium hexafluorophosphate (BOP) and the like, used in combination or singularly, may be utilized.

Furthermore, the instant conjugate may be formed by a non-peptidyl bond between the PSA cleavage site and a therapeutic agent. For example, the therapeutic agent may be covalently attached to the carboxyl terminus of the oligopeptide via a hydroxyl moiety on the therapeutic agent, thereby forming an ester linkage. For this purpose a reagent such as a combination of HBTU and HOBT, a combination of BOP and imidazole, a combination of DCC and DMAP, and the like may be utilized. The carboxylic acid may also be activated by forming the nitrophenyl ester or the like and reacted in the presence of DBU (1,8-diazabicyclo[5,4,0]undec-7-ene.

One skilled in the art understands that in the synthesis of compounds of the invention, one may need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to Protective Groups in Organic Chemistry, McOmie, ed., Plenum Press, NY, N.Y. (1973); and, Protective Groups in Organic Synthesis, Greene, ed., John Wiley & Sons, NY, N.Y. (1981) for the teaching of protective groups which may be useful in the preparation of compounds of the present invention.

By way of example only, useful amino-protecting groups may include, for example, C₁-C 10 alkanoyl groups such as formyl, acetyl, dichloroacetyl, propionyl, hexanoyl, 3,3-diethylhexanoyl, γ-chlorobutryl, and the like; C₁-C₁₀ alkoxycarbonyl and C₅-C₁₅ aryloxycarbonyl groups such as tert-butoxycarbonyl, benzyloxycarbonyl, allyloxycarbonyl, 4-nitrobenzyloxycarbonyl, fluorenylmethyloxycarbonyl and cinnamoyloxycarbonyl; halo-(C₁-C₁₀)-alkoxycarbonyl such as 2,2,2-trichloroethoxycarbonyl; and C₁-C₁₅ arylalkyl and alkenyl group such as benzyl, phenethyl, allyl, trityl, and the like. Other commonly used amino-protecting groups are those in the form of enamines prepared with β-keto-esters such as methyl or ethyl acetoacetate.

Useful carboxy-protecting groups may include, for example, C₁-C₁₀ alkyl groups such as methyl, tert-butyl, decyl; halo-C₁-C₁₀ alkyl such as 2,2,2-trichloroethyl, and 2-iodoethyl; C₅-C₁₅ arylalkyl such as benzyl, 4-methoxybenzyl, 4-nitrobenzyl, triphenylmethyl, diphenylmethyl; C₁-C₁₀ alkanoyloxymethyl such as acetoxymethyl, propionoxymethyl and the like; and groups such as phenacyl, 4-halophenacyl, allyl, dimethylallyl, tri-(C₁-C₃ alkyl)silyl, such as trimethylsilyl, β-p-toluenesulfonylethyl, β-p-nitrophenylthioethyl, 2,4,6-trimethylbenzyl, β-methylthioethyl, phthalimidomethyl, 2,4-dinitro-phenylsulphenyl, 2-nitrobenzhydryl and related groups.

Similarly, useful hydroxy protecting groups may include, for example, the formyl group, the chloroacetyl group, the benzyl group, the benzhydryl group, the trityl group, the 4-nitrobenzyl group, the trimethylsilyl group, the phenacyl group, the tert-butyl group, the methoxymethyl group, the tetrahydropyranyl group, and the like.

With respect to the preferred embodiment of an oligopeptide combined with vinblastine or desacetylvinblastine, the following Reaction Scheme illustrates the synthsis of the conjugates of the instant invention.

Reaction Scheme I illustrates preparation of conjugates of the cleavable oligopeptides and antimycin analog (represented above by Formula I) wherein the attachment of the antimycin (such as Antimycin A₃) is via the linker to the C-terminus of the oligopeptide. Addition of a single amino acid to the phenol prior to the incorporation of the remaining peptide portion of the oligopeptide may be advantageous if the functionality of the amino acids that comprise the oligopeptide would compete with the nucleophillic hydroxyl moiety. Alternatively, if no such competing functional groups are present on the oligopeptide, the oligopeptide may be attached to the linker in a single reaction step.

Reaction Scheme II illustrates incorporation of the cleavable oligopeptide on the antimycin analogs via a hydroxy alkyl carbonyl linker. The initial attachment is of the linker with a single N-protected amino acid, followed by deprotection and incorporation of the remaining fragment of the oligopeptide.

Reaction Scheme III illustrates incorporation of a cleavable oligopeptide into a therapeutic agent of the Formula II that has a phenol substituent.

Reaction scheme IV illustrates incorporation of a cleavable oligopeptide into a therapeutic agent of Formula IV that contains an amine moiety.

Reaction scheme V illustrates incorporation of a cleavable oligopeptide into a therapeutic agent of Formula V.

Reaction scheme VI illustrates incorporation of a cleavable oligopeptide into a therapeutic agent of Formula VI.

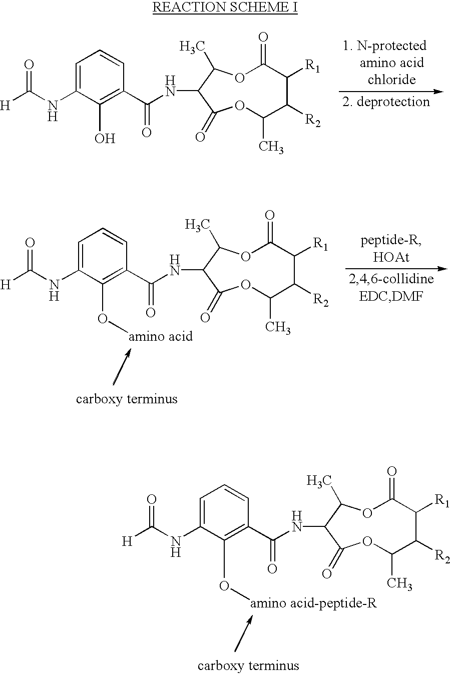

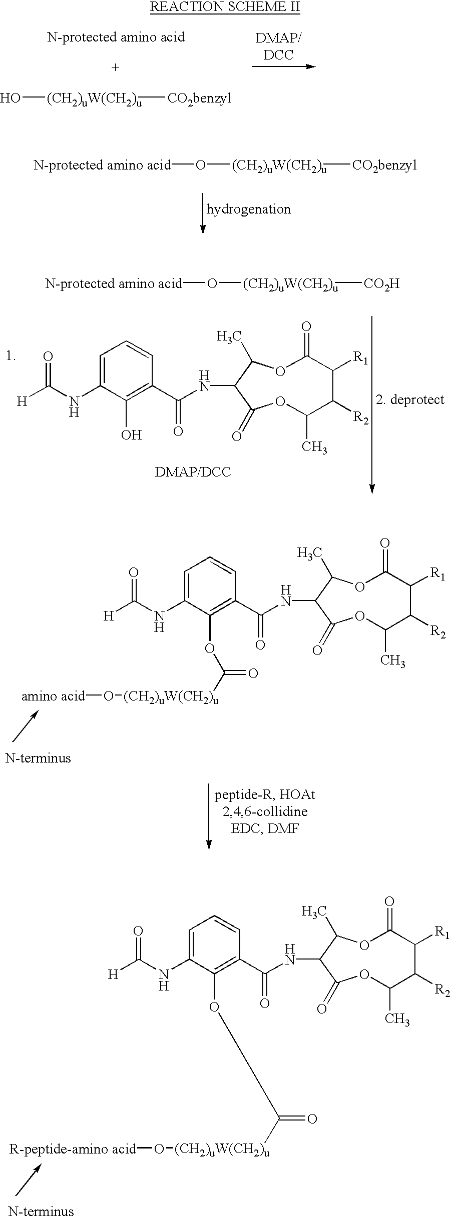

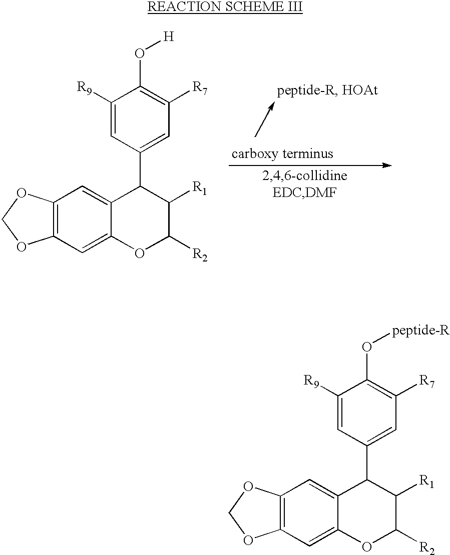

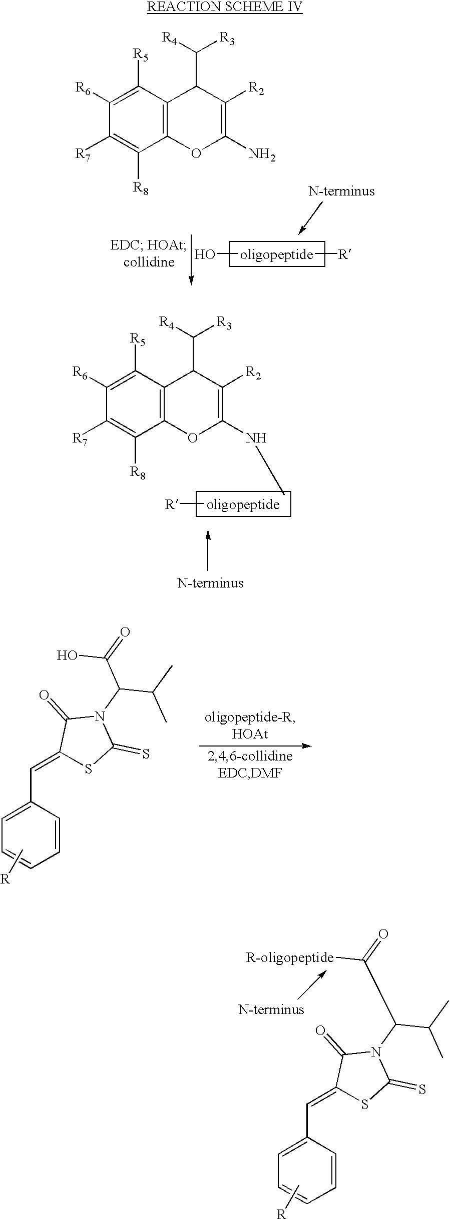

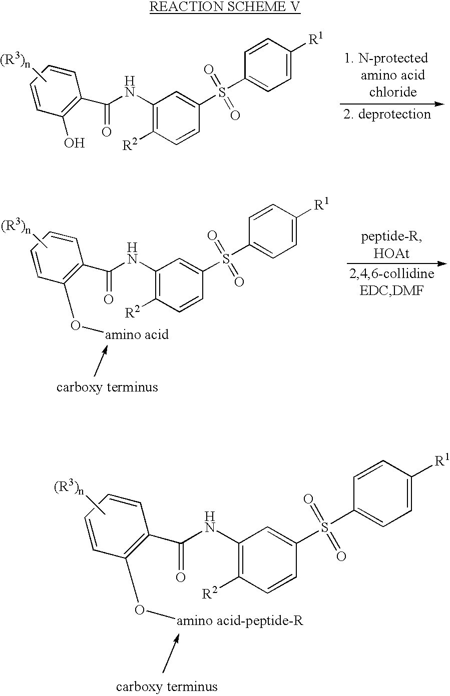

One skilled in the art will appreciate that although specific reagents and reaction conditions are outlined in the following examples, modification can be made which are meant to be encompassed by the spirit and scope of the invention. The following preparations and examples, therefore, are provided to further illustrate the invention, and are not limiting.

### EXAMPLES

### Example 1

Preparation of Compound 3

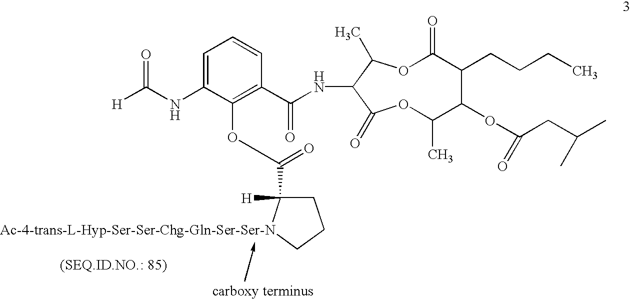

Step 1: Preparation of Compound 2

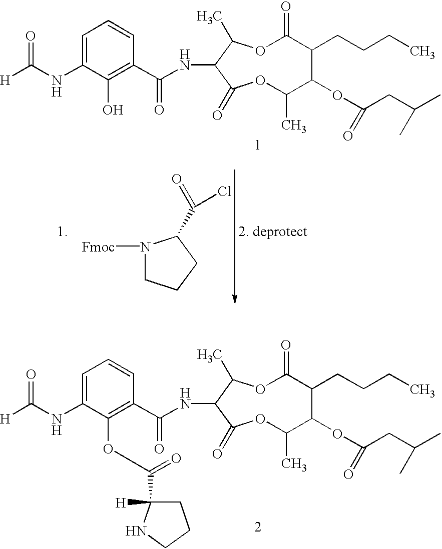

Compound 1 (1.0 mmol), dissolved in 3 ml of CH₂Cl₂ and 18 ml of anhydrous pyridine under nitrogen, is treated with 1.39 g of Fmoc-proline acid chlorine (Fmoc-Pro-Cl, Advanced Chemtech), and the mixture is stirred for 20 hr at 25° c. The progess of the reaction is monitored by HPLC and addition Fmoc-Pro-Cl is added as needed to complete the reaction. Upon complete disappearance of Compound 1, water (ca. 3 ml) is added to react with the excess aid chloride, and the solution is then evaporated to dryness and partitioned between 300 ml of EtOAc and 150 ml of saturated NaHCO₃, followed by washing twice with saturated NaCl. After drying (Na₂SO₄), the solvent is removed under reduced pressure to provide the N-protected prolyl ester, to which is added 30 ml of DMF and 14 ml of piperidine. After 5 min the solution is evaporated under reduced pressure to provide Compound 2. The crude product is dissolved in an acidic aqueous solution and washed with ether. The product is then purified by reverse phase HPLC to provide Compound 2 as a salt.

Step 2: N-Acetyl-4-trans-L-Hyp-Ser-Ser-Chg-Gln-Ser-Ser-WANG Resin (SEQ.ID.NO.: 85)

Starting with 0.5 mmole (0.61 g) of Fmoc-Ser(t-Bu)-WANG resin loaded at 0.82 mmol/g, the protected peptide was synthesized on a ABI model 430A peptide synthesizer adapted for Fmoc/t-butyl-based synthesis. The protocol used a 2-fold excess (1.0 mmol) of each of the following protected amino acids: Fmoc-Ser(t-Bu)-OH, Fmoc-Gln-OH, Fmoc-Chg-OH, Fmoc-4-trans-L-Hyp-OH; and acetic acid (double coupling). During each coupling cycle Fmoc protection was removed using 20% piperidine in N-methyl-2-pyrrolidinone (NMP), followed by washing with NMP. Coupling was achieved using DCC and HOBt activation in NMP. At the completion of the synthesis, the peptide resin was dried to yield the title compound.

Step 3: N-Acetyl-4-trans-L-Hyp-Ser-Ser-Chg-Gln-Ser-Ser-OH (SEQ.ID.NO.: 85)

One 0.5-mmol run of the above peptide-resin was suspended in 25 ml of TFA, followed by addition of 0.625 ml each of H₂O and triisopropylsilane, then stirring at 25° for 2.0 hr. The cleavage mixture was filtered, the solids were washed with TFA, the solvents were removed from the filtrate under reduced pressure, and the residue was triturated with ether to give a pale yellow solid, which was isolated by filtration and drying in vacuo to afford the title compound.

HPLC conditions, system A:

| | |
|---|---|
| Column . . . | Vydac 15 cm #218TP5415, C18 |
| Eluant . . . | Gradient (95%A → 50%A) over 45 min. |
| | A = 0.1% TFA/H₂O, B = 0.1% |
| | TFA/acetonitrile |
| Flow . . . | 1.5 ml/min. |

High Resolution ES/FT-MS: 789.3

Step 4: Compound 3

Samples of 522 mg (0.66 mmol) of the peptide from step 3 and ca. 0.6 mmol Compound 2 from Step 1, prepared as above, are dissolved in 17 ml of DMF under N₂. Then 163 mg (1.13 mmol) of 1-hydroxy-7-azabenzotriazole (HOAt) is added, and the pH is adjusted to 6.5-7 (moistened 5-10 range pH paper) with 2,4,6-collidine, followed by cooling to 0° C. and addition of 155 mg (0.81 mmol) of 1-(3-dimethylaminopropyl)-3-ethylcarbodiimide hydrochloride (EDC). Stirring is continued at 0-5° C. until completion of the coupling as monitored by analytical HPLC, maintaining the pH at 6.5-7 by periodic addition of 2,4,6-collidine. After 12 hr the reaction is worked up by addition of ˜4 ml of H₂O and, after stirring 1 hr, concentrated to a small volume in vacuo and dissolution in ca. 150 ml of 5% HOAc. The crude product is purified on preparative HPLC. Homogeneous fractions containing the desired product are pooled and concentrated to a volume of ˜50 ml and passed through approx. 40 ml of AG4×4 ion exchange resin (acetate cycle), followed by freeze-drying to provide Compound 1.

### Example 2

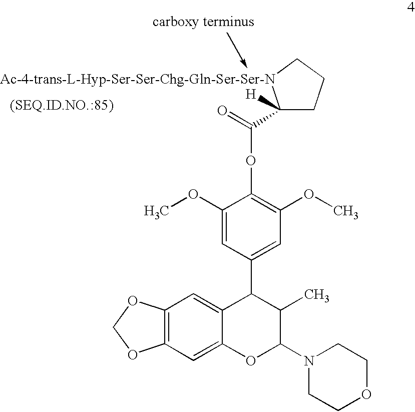

Compound 4 is prepared by the method described in Example 1, but substituting 4-[7,8-dihydro-7-methyl-6-(4-morpholinyl)-6H-1,3-dioxolo [4,5-g][1]benzopyran-8-yl]-2,6-dimethoxy-phenol, for Compound 1 in the Step 1.

### Example 3

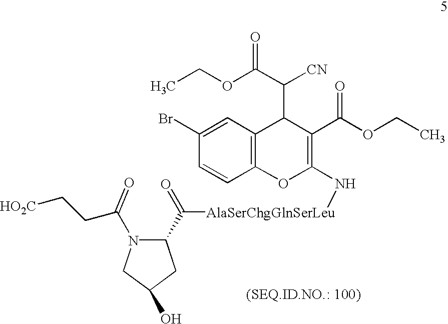

Preparation N-(N′-(Fm-Glutaryl)-trans-4-hydroxy-L-prolinyl-alanyl)serine

Step 1: N-Boc-trans-4-hydroxy-L-proline

A solution of trans-4-hydroxy-L-proline (3.0 kg, 22.88 M) in 1 M aqueous sodium hydroxide (25.2 L) and tert-butanol (12.0 L) was treated with a solution of di-tert-butyldicarbonate (5.09 kg) in tert-butanol (6.0 L) at 20° C. over 20 minutes. Upon complete addition, the resulting solution was stirred at 20° C. for 2 hours. The solution was extracted with hexane (2×15.0 L) and then acidified to pH 1 to 1.5 by cautious addition of a solution of potassium hydrogen sulphate (3.6 kg) in water (15.0 L). The mixture was extracted with ethyl acetate (3×15.0 L). The combined ethyl acetate extracts were washed with water (2×1.0 L) and dried by azeotropic distillation at atmospheric pressure (final KF of ethyl acetate solution <0.1%).

The ethyl acetate solution was then concentrated by atmospheric distillation to a volume of 15.0 L, diluted with hexane (8.0 L), seeded and stirred at 20° C. for 1 hour. Hexane (22.5 L) was added over 2 hours, the slurry was cooled to 0° C. for 1 hour and the solid collected by filtration. The product was washed with cold (0° C.) 2:1 hexane/ethyl acetate (15.0 L) and dried in vacuo at 45° C. to afford the title compound as a white crystalline solid.

Step 2: N-Boc-trans-4-hydroxy-L-proline Pentafluorophenyl Ester

Boc-trans-4-hydroxy-L-proline (3.5 kg) (prepared as described in Step 1) and pentafluorophenol (3.06 kg) were dissolved in ethyl acetate (52 L). The solution was treated with a solution of dicyclohexylcarbodiimide (3.43 kg) in ethyl acetate (8 L) and the mixture was stirred at room temperature for 2 hours. The resulting slurry was cooled to 0° C., filtered and the solids washed with ethyl acetate (15 L). The filtrate was evaporated at atmospheric pressure to a volume of 10 L and diluted with hexane (100 L). The resulting mixture was stirred at room temperature overnight and then cooled to 0° C. for 1 hour. The solid was collected by filtration, washed with cold (° C.) 10:1 hexane/ethyl acetate (15 L) and dried at 45° C. in vacuo to afford the title compound as a white crystalline solid.

Step 3: N-(trans-4-hydroxy-L-prolinyl-alanyl)serine Hydrochloride

N-alanylserine (1.5 kg, 8.515 M) and Boc-trans-4-hydroxy-L-proline pentafluorophenyl ester (3.72 kg) (prepared as described in step 2) were heated at 50° C. in dimethylformamide (15 L) for 3 hours. The solution was cooled to 20° C., treated with concentrated hydrochloric acid (7.5 L) and stirred at room temperature for 24 hours. The resulting slurry was diluted with isopropanol (30 L), stirred at room temperature for 30 minutes and then cooled to 0° C. for 1 hour. The solid was collected by filtration and washed with isopropanol (20 L). The solid was dried in vacuo at 40° C. to afford the title compound as a white crystalline solid.

Step 4: Fluorenylmethyl Glutarate

9-Fluorenyl methanol (2.0 kg), glutaric anhydride (2.33 kg) and sodium bicarbonate (1.71 kg) were stirred together in N-methylpyrrolidinone (8.0 L) at room temperature for 72 hours. The slurry was filtered and the solids washed with isopropyl acetate (2×10.0 L). The filtrate was washed with 1.0 M hydrochloric acid (3×10.0 L). The organic layer was extracted with 1.0 M aqueous sodium hydroxide (3×8.0 L). The combined basic extracts were covered with isopropyl acetate (20.0 L) and acidified to pH 2 with 2.0 M hydrochloric acid (12.5 L). The phases were separated and the aqueous phase was extracted with isopropyl acetate (10.0 L).

The combined organic phases were washed with water (10.0 L) and dried by azeotropic distillation at<60° C. under reduced pressure (KF<0.05%). The solution was then concentrated under reduced pressure (<60° C.) to a volume of 7.0 L. The solution was diluted with hexane (6.0 L), seeded and stirred at room temperature for 30 minutes. The resulting slurry was diluted by addition of hexane (42.0 L) over 40 minutes. The slurry was cooled to 0° C. for 1 hour and the solid collected by filtration and washed with cold (0° C.) 8:1 hexane/iPAC (20.0 L). The solid was dried in vacuo at 45° C. to afford the title compound as a pale cream solid.

Step 5: Fluorenylmethyl Glutarate Pentafluorophenyl Ester

Fluorenylmethyl glutarate (2.5 k g) (prepared as described in Step 4) and pentafluorophenol (1.63 kg) were dissolved in ethyl acetate (25 L). The solution was treated with a solution of dicyclohexylcarbodiimide (1.83 kg) in ethyl acetate (7.5 L) and the mixture was stirred at 20° C. overnight. The resulting slurry was filtered and the solids were washed through with ethyl acetate (10 L). The filtrate was evaporated at atmospheric pressure to a volume of 7.5 L and diluted with hexane (75 L). The slurry was filtered at 60-65° C. then allowed to cool to room temperature and stirred overnight. The slurry was cooled to 0° C. for 1 hour, the solid collected by filtration and washed with 10:1 hexane/ethyl acetate (15 L). The solid was dried in vacuo at 45° C. to afford the title compound as a white crystalline solid.

Step 6: N-(N′-(Fm-Glutaryl)-trans-4-hydroxy-L-prolinyl-alanyl)serine

N-(trans-4-hydroxy-L-prolinyl-alanyl)serine hydrochloride_(2.3 kg) (prepared as described in Step 3) was suspended in dimethylformamide (22 L) and the slurry was treated with N-ethylmorpholine (911 ml) followed by a solution of fluorenylmethyl glutarate pentafluorophenyl ester (3.5 kg) (prepared as described in Step 5) in dimethylformamide (14 L). The mixture was heated at 50° C. for 3 hours and the resulting solution evaporated to residue under reduced pressure. The residue was partitioned between water (80 L) and tert-butyl methyl ether (34 L). The phases were separated and the aqueous layer was extracted with tert-butyl methyl ether (34 L). The aqueous solution was seeded and stirred at room temperature overnight. The solid was collected by filtration (slow) and washed with water (25 L). The damp filter cake was dissolved in isopropanol (90 L) with warming and the solution concentrated to half volume by distillation at atmospheric pressure. Additional portions of isopropanol (3×45 L) were added and the batch was concentrated to ca half volume by atmospheric distillation after addition of each portion (Final KF of liquors<0.5%). The slurry was diluted with isopropanol (23 L), stirred at 20° C. overnight, cooled to 0° C. for 1 hour and the solid collected by filtration. The cake was washed with isopropanol (20 L) and the solid dried in vacuo at 45° C. to afford the crude product as a white solid.

Step 7: Recrystallisation of N-(N′-(Fm-Glutaryl)-trans-4-hydroxy-L-prolinyl-alanyl)serine

N-(N′-(Fm-Glutaryl)-trans-4-hydroxy-L-prolinyl-alanyl)serine (3.4 kg) (prepared as described in Step 6) was dissolved in methanol (51 L) at reflux. The solution was filtered and concentrated by atmospheric distillation to a volume of 17 L (5 ml/g). The solution was diluted with ethyl acetate (102 L) allowed to cool to 20° C. and stirred overnight. The resulting slurry was cooled to 0° C. for 1 hour and the solid was collected by filtration. The cake was washed with cold (0° C.) 10:1 ethyl acetate/methanol (20 L) and dried in vacuo at 45° C. to afford the product as a white solid.

Step 8: N-(serinyl)leucine Benzyl Ester Hydrochloride

Leucine benzyl ester p-tosylate (1000 g) and HOBt (412 g) were slurried in isopropyl acetate (12 L). The mixture was cooled to 0° C. in an ice-bath and a slurry of sodium bicarbonate (469.7 g) in water (1 L), N-BOC-L-serine (573.6 g) in water (2 L) and EDC.HCl (560.2 g) in water (2 L) were added. The mixture was allowed to warm to 20° C. over 30 minutes and aged at 20° C. for 2 hours (<1 A % Leu-OBn remaining). If the reaction was not complete after 2 hours, further NaHCO₃ and EDC.HCl were added. The phases were separated and the organic layer was washed sequentially with saturated sodium bicarbonate (2×3.75 L), 0.5 M sodium hydrogen sulphate (2×3.75 L) and water (2×2.5 L).

The wet, isopropyl acetate solution was concentrated under reduced pressure to 3 L and the water content checked. (KF=0.12%. It is important that this solution is dry prior to the addition of hydrogen chloride in isopropyl acetate). The solution was transferred to a 20 L round bottom flask under a nitrogen atmosphere and cooled to 0° C. To the solution was added 3.6 M HCl in isopropyl acetate (7 L, 10 mol equiv. HCl). The product began to crystallise after 5 minutes. The reaction was aged at 0° C. for 1 hr, and then allowed to warm to room temperature.

The slurry was cooled to 0-5° C., diluted with heptane (2.5 L) and aged at 0° C. for 30 minutes. The product was collected by filtration, washed with cold isopropyl acetate/heptane (4:1) (2.5 L) and dried in vacuo at 35° C., with a nitrogen sweep.

Step 9: N-(N′-(Boc)-glutaminyl-serinyl)leucine Benzyl Ester

N-(serinyl)leucine benzyl ester hydrochloride (350 g) (prepared as described in Step 8), HOBt (157.7 g) and N-Boc-L-glutamine (262.5 g) were slurried in DMF (2.5 L) and the mixture was cooled to 0° C. N-Ethylmorpholine (245.5 g) and EDC.HCl (214 g) were added and the mixture was aged at 0° C. for 2.5 hours. Water (14.7 L) was added over 20 minutes and the white slurry aged at 0° C. for 1 hour. The product collected by filtration and washed with water (3.2 L). The cake was dried in the fume-hood overnight. The isolated N-BOC-Gln-Ser-Leu-OBn, which contained DMF and HOBt, was combined with a second batch of identical size, and swished in water (12 L) at 20° C. for 1 hour. The product was collected by filtration, washed with water (2.5 L) and air-dried in a fume-hood over the weekend. The batch was dried in vacuo, at 42° C., with a nitrogen bleed.

Step 10: N-(glutaminyl-serinyl)leucine Benzyl Ester Hydrochloride

N-(N′-(Boc)-glutaminyl-serinyl)leucine benzyl ester (715 g, 1.33 M) (prepared as described in Step 9) was suspended in iPAC (3.5 L) at room temperature. To the slurry was added a 3.8 M solution of HCl in iPAC (3.5 L, 13.3 M) whereupon all the solids dissolved. After a short time, the product crystallised. The mixture was stirred at room temperature for 3.75 hours when HPLC showed complete reaction. The slurry was diluted with iPAC (4.0 L), stirred for 1 hour at room temperature and the solid collected by filtration under nitrogen. The product is very hygroscopic in the presence of excess HCl and must be collected under dry nitrogen.

The cake was washed with iPAC (4.0 L), the solid dried on the filter under nitrogen for 2 hours and then dried in vacuo at 45° C.

Step 11: N-(N′-(Boc)-cyclohexylglycylglutaminyl-serinyl)leucine Benzyl Ester (SEQ.ID.NO.: 107)

N-(glutaminyl-serinyl)leucine benzyl ester hydrochloride (SEQ.ID.NO.: 107) (2.6 kg) (prepared as described in Step 10), N-Boc-L-cyclohexylglycine (1.414 kg) and HOBt hydrate (168 g) were dissolved in DMF (13.0 L). N-ethylmorpholine (1.266 kg, 11.0 M) and EDC hydrochloride (1.265 kg) were added and the mixture stirred at 20° C. for 3 hours. The solution was diluted with ethyl acetate (13.0 L) and water (26.0 L) added. The product precipitated and the slurry was stirred at room temperature for 1 hour. The solid was collected by filtration, washed with 1:1 ethyl acetate/water (60 L) dried on the filter under nitrogen for 24 hours and dried in vacuo at 45°. The title compound was obtained as a white solid.

Step 12: N-(cyclohexylglycyl-glutaminyl-serinyl)leucine Benzyl Ester Hydrochloride (SEQ.ID.NO.: 108)

N-(N′-(Boc)-cyclohexylglycylglutaminyl-serinyl)leucine benzyl ester (SEQ.ID.NO.: 108) (1850 g) (prepared as described in Step 11) was slurried in isopropyl acetate (3.2 L). The slurry was cooled to 0° C. in an ice bath and 3.8 M HCl/isopropyl acetate (3.7 L, 11.4 mol equiv.) was added over 5 minutes, maintaining the temperature between 8 and 10° C. The starting material had dissolved after 15-20 minutes. The solution was seeded and the reaction aged at 8-10° C. for 2 hrs, (<1A % N-Boc-tetrapeptide-OBn remaining). The batch was filtered, under a nitrogen blanket, washed with cold (10° C.) isopropyl acetate (4×3 L) then dried on the filter under nitrogen. The solid was dried in vacuo, at 40° C.

The crude N-(cyclohexylglycyl-glutaminyl-serinyl)leucine benzyl ester (SEQ.ID.NO.: 108) hydrochloride (2.2 Kg) was slurried in methanol (22.3 L) at room temperature. The batch was stirred for 1 hour and then ethyl acetate (44.6 L) was added over 30 minutes. The batch was cooled to 0-5° C., aged for one hour, then filtered and washed with cold (0-5° C.) methanol/ethyl acetate (6 L, 1:2). The solid was dried on the filter, under nitrogen, for 45 minutes and then dried in vacuo, at 40° C., with a nitrogen sweep.

The N-(cyclohexylglycyl-glutaminyl-serinyl)leucine benzyl ester (SEQ.ID.NO.: 108) hydrochloride (1.478 Kg) was slurried in methanol (14.8 L) at room and the batch stirred for 1 hr. Ethyl acetate (29.6 L) was added over 30 minutes, the batch was cooled to 0-5° C. and aged for an hour. The solid collected by filtration, washed with cold (0-5° C.) methanol/ethyl acetate (4.5 L, 1:2), dried on the filter for 45 minutes, under nitrogen, and then dried under vacuum, at 40° C. This material was then utilized in subsequent reactions.

Step 13: N-(N′-(Fm-Glutaryl)-trans-4-hydroxy-L-prolinyl-alanyl-serine-cyclohexylglycyl-glutaminyl-serinyl)leucine Benzyl_ester (SEQ.ID.NO.: 105)

N-(cyclohexylglycyl-glutaminyl-serinyl)leucine benzyl ester hydrochloride (500 g) (prepared as described in Step 12), N-(N′-(Fm-Glutaryl)-trans-4-hydroxy-L-prolinyl-alanyl)serine (490 g) (prepared as described in Example 1) and HOAt (160 g) were slurried in DMF (8.2 L) and cooled to 2° C. in an ice bath. N-ethylmorpholine (135 ml) was added followed by EDC.HCl (210 g). The mixture was stirred at 0-2° C. for 2 hours and sampled. HPLC showed 0.2A % tetrapeptide remaining. The reaction mixture was diluted with ethyl acetate (4 L) and transferred to a 30-gallon glass vessel through a 5μ in-line filter. The flask and lines were rinsed with ethyl acetate/DMF (1:1, 500 ml) and ethyl acetate (4 L). Water (16.4 L) was added over 25 minutes (temperature 11° C. to 23° C.) and the mixture stirred slowly, at 20° C., for 30 minutes. The product was collected by filtration, washed with water (3 L), ethyl acetate (1 L) and water (2×3 L), then dried on the filter under nitrogen, and dried in vacuo at 45° C.

Step 14: N-(N′-(Fm-Glutaryl)-trans-4-hydroxy-L-prolinyl-alanyl-serine-cyclohexylglycyl-glutaminyl-serinyl)leucine (SEQ.ID.NO.: 106)

N-(N′-(Fm-Glutaryl)-trans-4-hydroxy-L-prolinyl-alanyl-serine-cyclohexylglycyl-glutaminyl-serinyl)leucine benzyl ester (1.1 Kg) (prepared as described in Step 13) was dissolved in dimethylacetamide (7.8 L) containing methanesulphonic acid (93.5 ml). 5% Pd/C (110 g, 10 wt %), slurried in DMA (1.0 L), was added and the mixture hydrogenated at atmospheric pressure for 1 hour 40 minutes. The reaction mixture was sampled: HPLC showed no starting material remaining.

The reaction mixture was filtered through a pre-wetted (DMA) pad of hyflo (500 g) to remove the catalyst. The hyflo pad washed with DMA (2.2 L) and then ethyl acetate (5.5 L). The filtrate was diluted with ethyl acetate (5.5 L) and stirred for 15 minutes. Water (44 L) was added over 40 minutes and the batch age for 1 hour. The solid collected by filtration, washed with water (1×10 L, 3×20 L), dried on the filter under a nitrogen blanket and dried in vacuo at 45° C.

Step 15: N-(N′-(Fm-Glutaryl)-trans-4-hydroxy-L-prolinyl-alanyl-serine-cyclohexylglycyl-glutaminyl-serinyl)leucine Swish Purification

Crude N-(N′-(Fm-Glutaryl)-trans-4-hydroxy-L-prolinyl-alanyl-serine-cyclohexylglycyl-glutaminyl-serinyl)leucine (2.58 kg) (prepared as described in Step 14) was sieved.

The solid (2.56 Kg) was swished in ethyl acetate for 3 hours. The solid was collected by filtration, washed with ethyl acetate (26 L), dried on the filter under nitrogen and dried in vacuo at 40° C.

Step 16: Preparation of Compound 8 (FMOC ester) (SEQ.ID.NO.: 106)

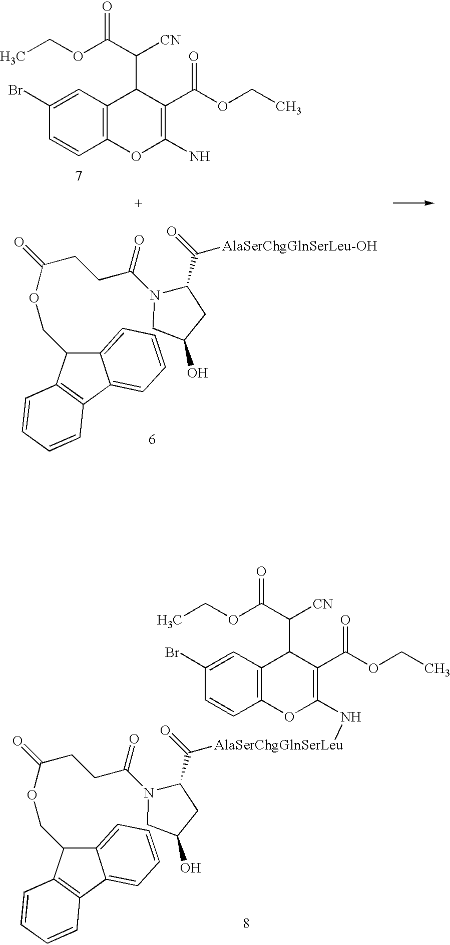

To a 3 necked, 12 L round bottom flask equipped with mechanical stirrer, thermocouple, and nitrogen inlet is charged DMF (5.1 L) and HOAt (319 mmoles). The yellow solution is inerted with nitrogen and warmed to 40° C. Heptapeptide 6 (266 mmoles) (prepared as described in Step 15) is added portion-wise to the warm solution; after stirring for 30 minutes at 40° C., a light yellow, opaque, homogeneous mixture results.

The mixture is cooled to room temperature, Compound 7 is added (274 mmoles), and resulting mixture is further cooled to −5° C. One equivalent of 2,4,6-collidine (35 ml) is added followed by 0.8 equivalents of EDC (213 mmoles) followed by the remaining two equivalents of 2,4,6-collidine (70 ml). The resulting slurry is aged at −5° C. to −3° C.

When the reaction has consumed starting heptapeptide 6, the reaction was quenched as follows.

In a 50 L, 4 necked round bottom flask equipped with a mechanical stirrer, thermocouple, and nitrogen inlet, is charged K₂HPO₄ (67.9 g), KH₂PO₄ (283 g), and water (13 L) to give a 0.19 M pH 6.3 buffer solution. The buffer solution is inerted with nitrogen, cooled to 15-18° C., and the cold reaction mixture (−1° C.) is added to the buffer via an addition funnel over 60 minutes maintaining the slurry temperature at 15-18° C. After complete addition, the resulting slurry is aged 15 minutes at 18° C., and filtered. The filter cake is displacement washed with water (1×6 L), followed by slurry washing with water (6×6 L), and drying in vacuo at room temperature with a nitrogen sweep. After drying for 48 hours, Compound 8 is obtained

Step 17: Preparation of Compound 5 (SEQ.ID.NO.: 100)

To a 3 necked, 12 L round bottom flask equipped with mechanical stirrer, thermocouple, and nitrogen inlet is charged Compound 8 (253.5 mmoles) and DMF (3.55 L). The solution is inerted with nitrogen, cooled to 1° C., and a solution of piperidine (404 mmoles, 1.6 eq.) in DMF (400 mL) is added drop-wise over 70 minutes maintaining the batch temperature at 0-2° C.

The reaction is monitored by HPLC and additional piperidine is charged as needed to complete the deprotection. The reaction is then quenched as follows.

In a 22 L, 3-necked round bottom flask equipped with mechanical stirrer, thermocouple, and nitrogen inlet is charged isopropyl acetate (12.1 L), inerted with nitrogen, and cooled to 0-5° C. To the cold i-PAc is added the cold (2° C.) reaction mixture via nitrogen pressure cannulation over 40 minutes. The resulting pink slurry is aged at 0-5° C. for thirty minutes then filtered under nitrogen. The cake is displacement washed with i-PAc (2×4 L), then slurry washed with i-PAc (3×4 L). All washes are done under a nitrogen blanket. The solid is dried in vacuo at room temperature with a nitrogen sweep for 24 hours to provide the Compound 5.

### Example 4

Assessment of the Recognition of Oligopeptide-Therapeutic Agent Drug Conjugates by Free PSA:

The conjugates prepared as described in Examples 1-3 are individually dissolved in PSA digestion buffer (50 mM tris(hydroxymethyl)-aminomethane pH7.4, 140 mM NaCl) and the solution added to PSA at a molar ration of 100 to 1. The reaction is quenched after various reaction times by the addition of trifluoroacetic acid (TFA) to a final 1% (volume/volume). Alternatively the reaction is quenched with 10 mM ZnCl12. The quenched reaction is analyzed by HPLC on a reversed-phase C18 column using an aqueous 0.1% TFA/acetonitrile gradient. The results are used to calculate the amount of time (in minutes) required for 50% cleavage of the noted oligopeptide-therapeutic agent conjugates with enzymatically active free PSA.

### Example 5

In vitro Assay of Cytotoxicity of Peptidyl Derivatives of Therapeutic Agent

The cytotoxicities of the the cleaveable oligopeptide-therapeutic agent conjugates, prepared as described in Examples 1-3, against a line of cells which expresses PSA. Specifically, cell cultures of LNCap prostate tumor cells, Colo320 DM cells (also designated C320), T24 bladder carcinoma cells or T47D breast carcinoma cells in 96 well plates were diluted with medium containing various concentrations of a given conjugate (final plate well volume of 200 μl). The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures.

### Example 7

In vivo Efficacy of Peptidyl-Therapeutic Agent Conjugates

LNCaP.FGC or C320 cells are trypsinized, resuspended in the growth medium and centifuged for 6 mins. at 200×g. The cells are resuspended in serum-free a-MEM and counted. The appropriate volume of this solution containing the desired number of cells is then transferred to a conical centrifuge tube, centrifuged as before and resuspended in the appropriate volume of a cold 1:1 mixture of a-MEM-Matrigel. The suspension is kept on ice until the animals are inoculated.

Harlan Sprague Dawley male nude mice (10-12 weeks old) are restrained without anesthesia and are inoculated with 0.5 mL of cell suspension on the left flank by subcutaneous injection using a 22 G needle. Mice are either given approximately 5×105 DuPRO cells or 1.5×107 LNCaP.FGC cells.

Following inoculation with the tumor cells the mice are treated under one of two protocols:

Protocol A:

One day after cell inoculation the animals are dosed with a 0.1-0.5 mL volume of test conjugate or vehicle control (sterile water). Dosages of the conjugate are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B:

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate or vehicle control (sterile water). Dosages of the conjugate are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

### Example 8

In vitro Determination of Proteolytic Cleavage of Conjugates by Endogenous non-PSA Proteases

Step A: Preparation of Proteolytic Tissue Extracts

All procedures are carried out at 4° C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a lose fitting and then a tight fitting pestle. The homogenate is centrifuged at 10,000×g in a swinging bucket rotor (HB4-5), the pellet is discarded and the re-supernatant centrifuged at 100,000×g (Ti 70). The supernatant (cytosol) is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000×g. The supernatant is discarded and the pellet resuspended in Buffer C (10 mM potassium phosphate buffer containing0.25 M sucrose, pH 7.4), using ½ the volume used above, and homogenized with a dounce homogenizer.

Protein content of the two solutions (cytosol and membrane) is determine using the Bradford assay. Assay aliquots are then removed and frozen in liquid N₂. The aliquots are stored at −70° C.

Step B: Proteolytic Cleavage Assay

For each time point, 20 microgram of conjugate and 150 micrograms of tissue protein, prepared as described in Step A and as determined by Bradford in reaction buffer are placed in solution of final volume of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C18 15 cm column in water/acetonitrile (5% to 50% acetonitrile over 30 minutes).

What is claimed is:

1. A conjugate which is useful for the treatment of prostate cancer which comprises an oligopeptide covalently bonded, either directly or through a chemical linker, to a peptide or small molecule that binds to an anti-apoptotic Bcl-2 family protein, inhibits the expression of the Bcl-2 family protein, or inhibits the function of the Bcl-2 family protein,
wherein the oligopeptide is an oligomer that is selectively recognized by enzymatically active prostate specific antigen (PSA) and is capable of being proteolytically cleaved by the enzymatic activity of the prostate specific antigen;
or the pharmaceutically acceptable salt thereof.

2. The conjugate according to claim 1, or the pharmaceutically acceptable salt thereof, wherein the oligopeptide comprises an amino acid sequence selected from:
a) AsnLysIleSerTyrGln|Ser (SEQ.ID.NO.: 1),
b) LysIleSerTyrGln|Ser (SEQ.ID.NO.: 2),
c) AsnLysIleSerTyrTyr|Ser (SEQ.ID.NO.: 3),
d) AsnLysAlaSerTyrGln|Ser (SEQ.ID.NO.: 4),
e) SerTyrGln|SerSer (SEQ.ID.NO.: 5);
f) LysTyrGln|SerSer (SEQ.ID.NO.: 6);
g) hArgTyrGln|SerSer (SEQ.ID.NO.: 7);
h) hArgChaGln|SerSer (SEQ.ID.NO.: 8);
i) TyrGln|SerSer (SEQ.ID.NO.: 9);
j) TyrGln|SerLeu (SEQ.ID.NO.: 10);
k) TyrGln|SerNle (SEQ.ID.NO.: 11);
l) ChgGln|SerLeu (SEQ.ID.NO.: 12);
m) ChgGln|SerNle (SEQ.ID.NO.: 13);
n) SerTyrGln|Ser (SEQ.ID.NO.: 14);
o) SerChgGln|Ser (SEQ.ID.NO.: 15);
p) SerTyrGln|SerVal (SEQ.ID.NO.: 16);
q) SerChgGln|SerVal (SEQ.ID.NO.: 17);
r) SerTyrGln|SerLeu (SEQ.ID.NO.: 18);
s) SerChgGln|SerLeu (SEQ.ID.NO.: 19);
t) HaaXaaSerTyrGln|Ser (SEQ.ID.NO.: 20);
u) HaaXaaLysTyrGln|Ser (SEQ.ID.NO.: 21);
v) HaaXaahArgTyrGln|Ser (SEQ.ID.NO.: 22);
w) HaaXaahArgChaGln|Ser (SEQ.ID.NO.: 23);
x) HaaTyrGln|Ser (SEQ.ID.NO.: 24);
y) HaaXaaSerChgGln|Ser (SEQ.ID.NO.: 25);
z) HaaChgGln|Ser (SEQ.ID.NO.: 26);
aa) SerChgGln|SerSer (SEQ.ID.NO.: 27);
bb) SerChgGln|SerPro (SEQ.ID.NO.: 28);
cc) SerChgGln|SerAbu (SEQ.ID.NO.: 29);
wherein Haa is a cyclic amino acid substituted with a hydrophilic moiety, hArg is homoarginine, Xaa is any amino acid, Cha is cyclohexylalanine, Abu is 2-aminobutyric acid and Chg is cyclohexylglycine.

3. The conjugate according to claim 1, or the pharmaceutically acceptable salt thereof, wherein the oligopeptide comprises an amino acid sequence selected from:
SerSerChgGln|SerLeu (SEQ.ID.NO.: 46);
SerSerChgGln|SerVal (SEQ.ID.NO.: 47);
SerSerChgGln|SerPro (SEQ.ID.NO.: 48);
SerSerChgGln|SerSer (SEQ.ID.NO.: 49);
SerSerSerChgGln|SerLeu (SEQ.ID.NO.: 50);
SerSerSerChgGln|SerVal (SEQ.ID.NO.: 51);
SerSerSerChgGln|SerPro (SEQ.ID.NO.: 52);
SerSerSerChgGln|SerSer (SEQ.ID.NO.: 53);
SerAlaSerChgGln|SerLeu (SEQ.ID.NO.: 54);
SerAlaSerChgGln|SerVal (SEQ.ID.NO.: 55);
(N-methyl-Ser)SerSerChgGln|SerLeu (SEQ.ID.NO.: 56);
(N-methyl-Ser)SerSerChgGln|SerVal (SEQ.ID.NO.: 57);
4-HypSerSerTyrGln|SerVal (SEQ.ID.NO.: 58);
4-HypSerSerTyrGln|SerLeu (SEQ.ID.NO.: 59);
4-HypSerSerChgGln|SerVal (SEQ.ID.NO.: 60);
4-HypSerSerChgGln|SerLeu (SEQ.ID.NO.: 61);
4-HypSerSerChgGln|SerSer (SEQ.ID.NO.: 62);
4-HypSerSerChgGln|SerSer (SEQ.ID.NO.: 63);
4-HypSerSerChgGln|SerPro (SEQ.ID.NO.: 64);
4-HypSerSerChgGln|SerPro (SEQ.ID.NO.: 65);
4-HypAlaSerChgGln|SerVal (SEQ.ID.NO.: 66);
4-HypAlaSerChgGln|SerLeu (SEQ.ID.NO.: 67);
(3,4-DiHyp)SerSerTyrGln|SerVal (SEQ.ID.NO.: 68); and
(3,4-DiHyp)SerSerTyrGln|SerLeu (SEQ.ID.NO.: 69);
wherein 4-Hyp is 4-hydroxyproline, 3,4-DiHyp is 3,4-dihydroxyproline and Chg is cyclohexylglycine.

4. The conjugate according to claim 1, or the pharmaceutically acceptable salt thereof, wherein the peptide or small molecule that binds to an anti-apoptotic Bcl-2 family protein or inhibits the expression of such a Bcl-2 family protein is a small molecule selected from:
i) ANTIMYCINS OF FORMULA (I): 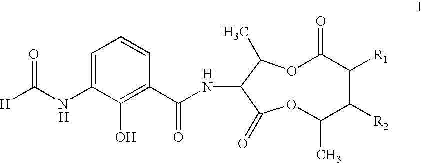
in which
R₁ is H or C₁-C₁₀ alkyl
R₂ is hydrogen, —OH, or —(C₁-C₁₀ alkyl)—CO₂H;
or a pharmaceutically acceptable salt or optical isomer thereof;
ii) compounds of the formula II as described in PCT Publ. No. WO 00/04901: 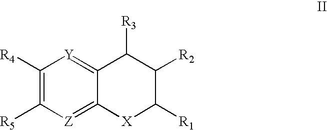
wherein:
X is selected from the group consisting of CH₂; CHOCH₃; NH; O; and S;
Y and Z are independently selected from the group consisting of CH and N; and when Z is N, then Y may further be —CR₆, where R₆ is selected from the group consisting of CH₃; OCH₃; CNH₂; and COH;
R₁ is selected from the group consisting of hydrogen; C₁₋₅alkyl; C₁₋₅alkoxy; OH; NH₂; NO₂; CHO; COCH₃; COOH; COOCH₃; N(C₁₋₃alkyl)₂; NH(C₁₋₃alkyl); OCOCH₃; OCOCH₂CH₃; NHCOCH₃; NHNHCOCH₃; NHNHCONH₂; phenyl; phenyl which is mono-, di-, or tri-substituted with NH₂, OH, halogen, NO₂, CF₃, COOH or COOCH₃; cyclohexyl; cyclohexyl which is mono- di-, or tri-substituted with NH₂, OH, halogen or CF₃; and five- and six-member heterocyclic rings, preferably a heterocyclic ring selected from the group consisting of piperidino, piperazino, morpholino, pyrimidyl, pyrrolidino and imidazo;
R₂ is selected from the group consisting of hydrogen; C₁₋₃alkyl C₁₋₃alkoxy; halogen; CF₃; NH₂; OH; COOH; COOCH₃; CONH₂; and CONHCH₃;
or R₁ and R₂ together may form the group —CH₂CH₂CH₂— or —CH₂CH₂CH₂CH₂—;
or R₁ and R₂ together may form, starting from R₁, the group —NHCH₂CH₂, —NHCOCH₂—, or —OCOCH₂—;
R₃ is selected from the group consisting of H; CH₃; CF₃; OCH₃; NH₂; OH; COOH; COCH₃; CH═CH₂; CH₂═CHCH₂; CH(CH₃)₂; CH₂OH; CH₂NH₂; CH₂COOH; cyclohexyl; cyclohexyl which is mono- di-, or tri-substituted with NH₂, OH, halogen, OCH₃ or CF₃; five- and six-member heterocyclic rings, preferably a heterocyclic ring selected from the group consisting of piperidinyl, piperazinyl, morpholino, pyrimidyl, pyrrolyl, pyrrolidino, and imidazyl; and a substituted phenyl group of the formula: 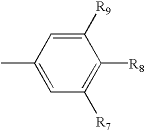
wherein
R₇, R₈ and R₉ are independently selected from the group consisting of hydrogen, CH₃, CF₃, OH, OCH₃, CH₂OH and CHO; provided that at least two of the members of the group R₇, R₈ and R₉ must be OH or OCH₃ when the remaining member of the group is hydrogen, CH₃ or CF₃;
R₄ and R₅ are independently selected from the group consisting of hydrogen, CH₃, and OCH₃; and when Y and Z are both CH, R₄ and R₅ may be further selected from OH and NH₂;
or, R₄ and R₅ together may form the group —CH₂CH₂CH₂— or —CH₂CH₂CH₂CH₂—;
or, R₄ and R₅ together may form, starting from R₄, the group —NHCH₂CH₂—, —NHCOCH₂—, OCOCH₂— or —O(CH₂)nO—, wherein n is 1, 2 or 3;
or a pharmaceutically acceptable salt thereof when the compound includes at least on NH₂ or COOH substituent;
iii) a compound of the formula III as described in PCT Publ. No. WO 00/04901: 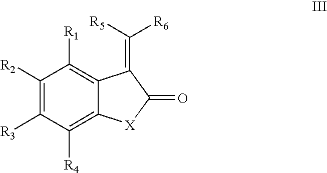
wherein
R₁, R₂, R₃ and R₄ are independently selected from the group consisting of hydrogen; C₁₋₅alkyl; C₁₋₅alkoxy; OH; NH₂; NO₂; CHO; COCH₃; COOH; COOCH₃; N(C₁₋₃alkyl)₂; and NH(C₁₋₃alkyl); and one of R₁, R₂, R₃ and R₄ may be phenyl or a heterocyclic ring, preferably a heterocyclic ring selected from the group consisting of piperidino, piperazino, morpholino, pyrimidyl, pyrrolidino and imidazo; provided at least one of R₁, R₂, R₃ and R₄ must be hydrogen;
R₅ and R₆ are independently selected from the group consisting of hydrogen; CN; CH₂CN; COOCH₃; CONH₂; phenyl; phenyl which is mono-, di-, or tri-substituted with NH₂, OH, halogen, NO₂, CH₃, OCH₃, CF₃, COOH or COOCH₃; cyclohexyl; cyclohexyl which is mono-, di-, or tri-substituted with NH₂, OH, halogen or CF₃; and five- and six-member heterocyclic rings, preferably a heterocyclic ring selected from the group consisting of pyrrolyl, imidazolyl, piperidinyl, piperazinyl, morpholino, pyrimidyl and pyrrolidino; provided, only one of R₅ or R₆ may be phenyl, substituted phenyl, cyclohexyl, substituted cyclohexyl or heterocyclic in the same compound, and further provided that when one of R₅ or R₆ is phenyl, substituted phenyl, cyclohexyl, substituted cyclohexyl or heterocyclic, then the other must be hydrogen;
or at least one of R₅ and R₆ may be halogen, provided that the other must be C₁₋₅alkyl or C₁₋₅alkoxy;
or a pharmaceutically acceptable salt thereof when the compound included at least one NH₂ or COOH substituent;
iv) a compound of the formula IV as described in PCT Publ. No. WO 00/04901: 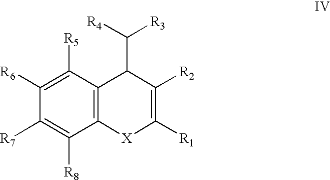
wherein:
X is selected from the group consisting of CH₂; CHOCH₃; NH; NCH₃; O; and S;
R₁ is selected from the group consisting of OH; NH₂; CHO; COCH₃; COOH; N(C₁₋₃alkyl); OCOCH₃; OCOCH₂CH₃; NHCOCH₃; NHNHCOCH₃; NHNHCONH₂; N(C₁₋₃alkyl)₂; NH(C₁₋₃alkyl); and five- and six-member heterocyclic rings, preferably a heterocyclic ring selected form the group consisting piperidinyl, piperazinyl, morpholino, pyrimidyl, pyrrolyl, pyrrolidino and imidazyl;
R₂ is selected from the group consisting of C₁₋₃alkyl; C₁₋₃alkoxy; OH: NH₂; CHO; COCH₃; OCOCH₃; OCOCH₂CH₃; COOH; COOCH₃; COOCH₂CH₃; COOCH₂CH₂CH₃;
R₃ is selected from the group consisting of C₁₋₃alkyl; C₁₋₃alkoxy; CN; CH₂CN; CH₂NO₂; CHO; COCH₃; COOH; OCOCH₃; OCOCH₂CH₃; NHCOCH₃; NHNHCOCH₃; NHNHCONH₂; CH═CH₂; CH₂CH═CH₂; CH₂CHO; and five- and six-member heterocyclic rings, preferably a heterocyclic ring selected from the group consisting piperidinyl, piperazinyl, morpholino, pyrimidyl, pyrrolyl, pyrrolidino and imidazyl;
R₄ is selected from the group consisting of C₁₋₃alkyl, C₁₋₃alkoxy; CN; CH₂CN; CH₂NO₂; CHO; COCH₃; COCH₃; COOH; COOCH₃; COOCH₂CH₃; COOCH₂CH₂CH₃; OCOCH₃; OCOCH₂; CH₃;
R₅ is selected from the group consisting of hydrogen CH₃; OCH₃; OH; NH₂; Br; Cl; and F; and
R₆, R₇ and R₈ are selected from the group consisting of hydrogen, CH₃; CH₂CH₃; CF₃; NH₂; OH; OCH₃; CN; NO₂; CL; Br; F; COOH; and COOCH₃; provided, at least one member of the group R₆, R₇ or R₈ must be Cl, Br or F when the remaining members of said group are hydrogen;
or a pharmaceutically acceptable salt thereof when the compound includes at least one NH₂ or COOH substituent;
v) a compound of the formula V 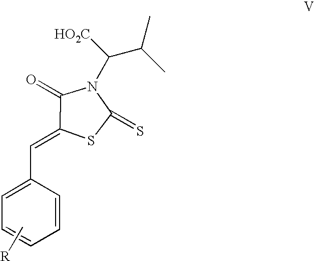
wherein R is selected from H, halogen, NH₂, NH(C₁-C₆ alkyl) and N(C₁-C₆ alkyl)₂; or a pharmaceutically acceptable salt or optical isomer thereof; and
vi) a compounds of the formula VI: 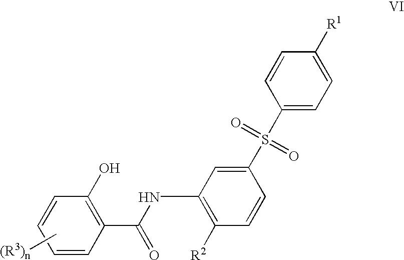
wherein:
R¹ is halogen;
R² is halogen;
R³ is halogen; and
n is 0, 1 or 2
or a pharmaceutically acceptable salt or optical isomer thereof.

5. The conjugate according to claim 4 wherein the small molecule is selected from:

| | |
|---|---|
| | I |
| 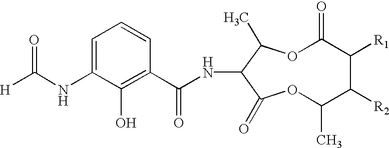 | |
| Name | R₁ | R₂ |
|---|---|---|
| actimycin A₀₍ₐ₎ | hexyl | hexanoic acid |
| actimycin A_{0(b)} | butyl | heptanoic acid |
| antimycin A_{0(c)} | octyl | pentanoic acid |
| antimycin A_{0(d)} | heptyl | pentanoic acid |
| antimycin A₁ | hexyl | isovaleric acid |
| antimycin A₂ | hexyl | butanoic acid |
| antimycin A₃ | butyl | isobutanoic acid |
| antimycin A₄ | butyl | butanoic acid |
| antimycin A₅ | ethyl | isobutanoic acid |
| antimycin A₆ | ethyl | butanoic acid |
| kitamycin A | hexyl | hydroxyl |
| kitamycin B | isohexyl | hydroxyl |
| urauchimycin B | isohexyl | hydroxyl |
| deisovalerylblastomycin | butyl | hydroxyl |
| dehexyl-deisovalerylblastomycin | hydrogen | hydrogen |

6. The conjugate according to claim 4 wherein the small molecule is selected from:

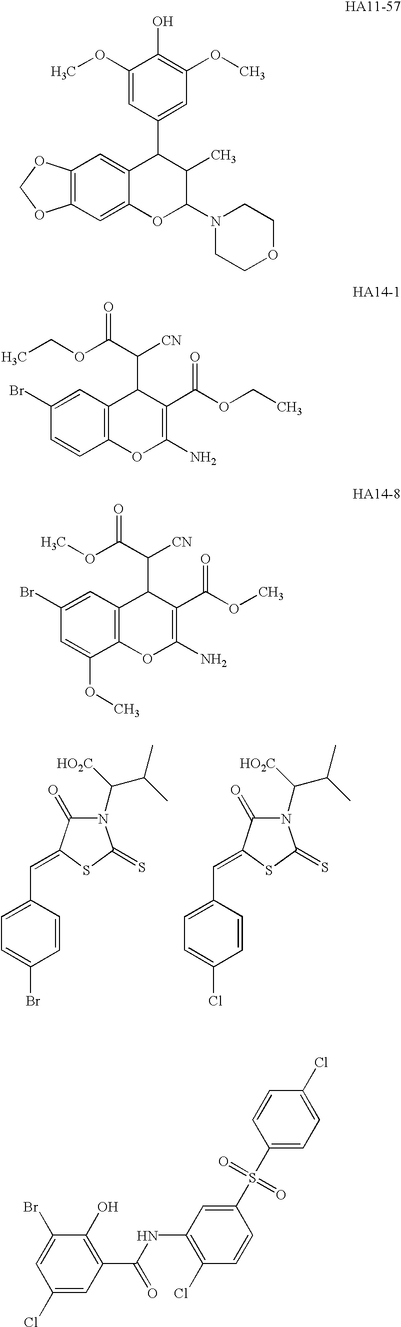

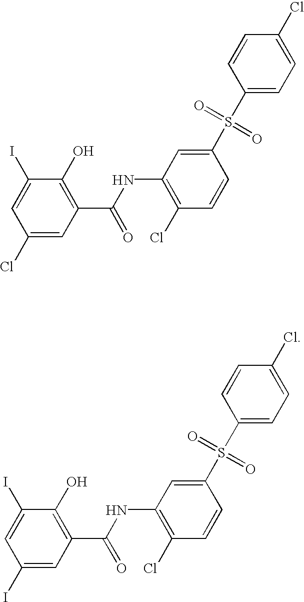

7. The conjugate according to claim 1 of the formula A: 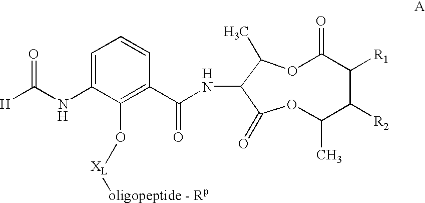
wherein:
oligopeptide is an oligopeptide which is selectively recognized by enzymatically active prostate specific antigen (PSA) and is capable of being proteolytically cleaved by the enzymatic activity of the prostate specific antigen,
X_{L} is selected from: a bond, —C(O)—(CH₂)ᵤ—W—(CH₂)ᵤ—O—0 and —C(O)—(CH₂)ᵤ—W—(CH₂)ᵤ—NH—;
R₁ is H or C₁-C₁₀ alkyl;
R₂ is hydrogen, —OH, or —(C₁-C₁₀ alkyl)—CO₂H;
R^{p} is selected from
a) hydrogen,
b) —(C═O)R^{a}, 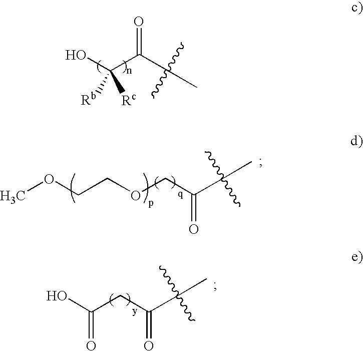
f) ethoxysquarate; and
g) cotininyl;
R^{b} and R^{c} are independently selected from:
a) hydrogen,
b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,
c) unsubstituted C₁-C₆ alkyl,
d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or
R^{b} and R^{c} are combined to form —(CH₂)ₛ— wherein one of the carbon atoms is optionally replaced by a moiety selected from: O, S(O)ₘ, —NC(O)—, NH and —N(COR^{e})—,
R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,
R^{d} is selected from: hydrogen, aryl, substituted aryl, heterocycle, substituted heterocycle, C₁-C₆ alkyl and C₃-C₁₀ cycloalkyl;
R^{e} is selected from: aryl, substituted aryl, heterocycle, substituted heterocycle, C₁-C₆ alkyl and C₃-C₁₀ cycloalkyl;
W is selected from a bond, a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
u is selected from: 0, 1, 2 or 3;
y is 1, 2 or 3;
or the pharmaceutically acceptable salt thereof.

8. The conjugate according to claim 1 of the formula B: 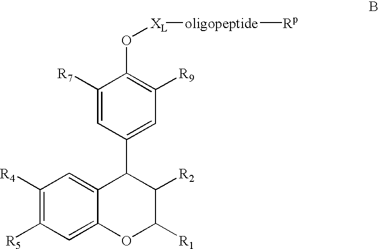
wherein:
oligopeptide is an oligopeptide which is selectively recognized by enzymatically active prostate specific antigen (PSA) and is capable of being proteolytically cleaved by the enzymatic activity of the prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R₁ is selected from the group consisting of hydrogen; C₁₋₅alkyl; C₁₋₅alkoxy; OH; NH₂; NO₂; CHO; COCH₃; COOH; COOCH₃; N(C₁₋₃alkyl)₂; NH(C₁₋₃alkyl); OCOCH₃; OCOCH₂CH₃; NHCOCH₃; NHNHCOCH₃; NHNHCONH₂; phenyl; phenyl which is mono-, di-, or tri-substituted with NH₂, OH, halogen, NO₂, CF₃, COOH or COOCH₃; cyclohexyl; cyclohexyl which is mono- di-, or tri-substituted with NH₂, OH, halogen or CF₃; and five- and six-member heterocyclic rings, preferably a heterocyclic ring selected from the group consisting of piperidino, piperazino, morpholino, pyrimidyl, pyrrolidino and imidazo;
R₂ is selected from the group consisting of hydrogen; C₁₋₃alkyl C₁₋₃alkoxy; halogen; CF₃; NH₂; OH; COOH; COOCH₃; CONH₂; and CONHCH₃;
or R₁ and R₂ together may form the group —CH₂CH₂CH₂— or —CH₂CH₂CH₂CH₂—;
or R₁ and R₂ together may form, starting from R₁, the group —NHCH₂CH₂, —NHCOCH—, or —OCOCH₂—;
R₇ and R₉ are independently selected from the group consisting of hydrogen, CH₃, CF₃, OH, OCH₃, CH₂OH and CHO; provided that at least of R₇ and R₉ must be OH or OCH₃ when the remaining member of the group is hydrogen, CH₃ or CF₃;
R^{p} is selected from
a) hydrogen,
b) —(C═O)R^{a}, 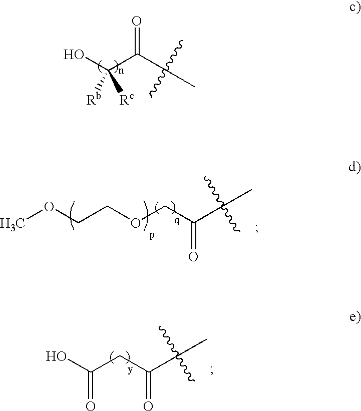
f) ethoxysquarate; and
g) cotininyl;
R^{b} and R^{c} are independently selected from:
a) hydrogen,
b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,
c) unsubstituted C₁-C₆ alkyl,
d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or
R^{b} and R^{c} are combined to form —(CH₂)ₛ— wherein one of the carbon atoms is optionally replaced by a moiety selected from: O, S(O)ₘ, —NC(O)—, NH and —N(COR^{e})—;
R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,
R^{d} is selected from: hydrogen, aryl, substituted aryl, heterocycle, substituted heterocycle, C₁-C₆ alkyl and C₃-C₁₀ cycloalkyl;
R^{e} selected from: aryl, substituted aryl, heterocycle, substituted heterocycle, C₁-C₆ alkyl and C₃-C₁₀ cycloalkyl;
W is selected from a bond, a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
u is selected from: 0, 1, 2 or 3;
y is 1, 2 or 3;
or a pharmaceutically acceptable salt thereof.

9. The conjugate according to claim 1 of the formula C: 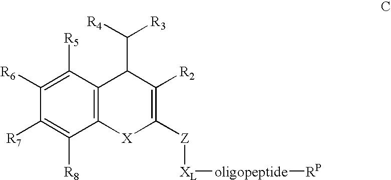
wherein:
oligopeptide is an oligopeptide which is selectively recognized by enzymatically active prostate specific antigen (PSA) and is capable of being proteolytically cleaved by the enzymatic activity of the prostate specific antigen,
X is selected from the group consisting of CH₂, CHOCH₃, NH, NCH₃, O and S;
X_{L} is selected from: a bond, —C(O)—(CH₂)ᵤ—W—(CH₂)ᵤ—O— and —C(O)—(CH₂)ᵤ—W—(CH₂)ᵤ—NH—;
Z is selected from: —O— and —NH—;
R₂ is selected from the group consisting of C₁₋₃alkyl; C₁₋₃alkoxy; OH: NH₂; CHO; COCH₃; OCOCH₃; OCOCH₂CH₃; COOH; COOCH₃; COOCH₂CH₃; COOCH₂CH₂CH₃;
R₃ is selected from the group consisting of C₁₋₃alkyl; C₁₋₃alkoxy; CN; CH₂CN; CH₂NO₂; CHO; COCH₃; COOH; OCOCH₃; OCOCH₂CH₃; NHCOCH₃; NHNHCOCH₃; NHNHCONH₂; CH═CH₂; CH₂CH═CH₂; CH₂CHO; and five- and six-member heterocyclic rings, preferably a heterocyclic ring selected from the group consisting piperidinyl, piperazinyl, morpholino, pyrimidyl, pyrrolyl, pyrrolidino and imidazyl;
R₄ is selected from the group consisting of C₁₋₃alkyl, C₁₋₃alkoxy; CN; CH₂CN; CH₂NO₂; CHO; COCH₃; COCH₃; COOH; COOCH₃; COOCH₂CH₃; COOCH₂CH₂CH₃; OCOCH₃; OCOCH₂, CH₃;
R₅ is selected from the group consisting of hydrogen CH₃; OCH₃; OH; NH₂; Br; Cl; and F; and
R₆, R₇ and R₈ are selected from the group consisting of hydrogen, CH₃; CH₂CH₃; CF₃; NH₂; OH; OCH₃; CN; NO₂; CL; Br; F; COOH; and COOCH₃; provided, at least one member of the group R₆, R₇ or R₈ must be Cl, Br or F when the remaining members of said group are hydrogen;
R^{p} is selected from
a) hydrogen,
b) —(C═O)R^{a}, 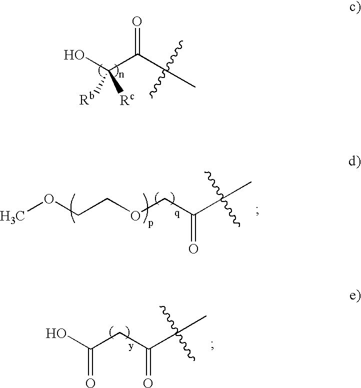
f) ethoxysquarate; and
g) cotininyl;
R^{b} and R^{c} are independently selected from:
a) hydrogen,
b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,
c) unsubstituted C₁-C₆ alkyl,
d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or
R^{b} and R^{c} are combined to form —(CH₂)ₛ— wherein one of the carbon atoms is optionally replaced by a moiety selected from: O, S(O)ₘ, —NC(O)—, NH and —N(COR^{e})—;
R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,
R^{d} is selected from: hydrogen, aryl, substituted aryl, heterocycle, substituted heterocycle, C₁-C₆ alkyl and C₃-C₁₀ cycloalkyl;
R^{e} is selected from: aryl, substituted aryl, heterocycle, substituted heterocycle, C₁-C₆ alkyl and C₃-C₁₀ cycloalkyl;
W is selected from a bond, a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
u is selected from: 0, 1, 2 or 3;
y is 1, 2 or 3;
or the pharmaceutically acceptable salt thereof.

10. The conjugate according to claim 1 of the formula D: 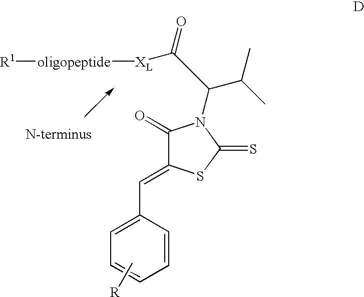
wherein:
oligopeptide is an oligopeptide which is selectively recognized by enzymatically active prostate specific antigen (PSA) and is capable of being proteolytically cleaved by the enzymatic activity of the prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R is selected from H, halogen, NH₂, NH(C₁-C₆ alkyl) and N(C₁-C₆ alkyl)₂; and
R¹ is selected from H, C₁-C₁₈ alkyl, benzyl, aryl, NH₂, NH(C₁-C₆ alkyl), N(C₁-C₆ alkyl)₂, morpholinyl and piperidinyl;
W is selected from a bond, a branched or straight chain C₁-C₆-alkyl, cyclopentyl, cyclohexyl, cycloheptyl or bicyclo[2.2.2]octanyl;
u is selected from: 0, 1, 2 or 3;
or the pharmaceutically acceptable salt thereof.

11. The conjugate according to claim 1 of the formula E: 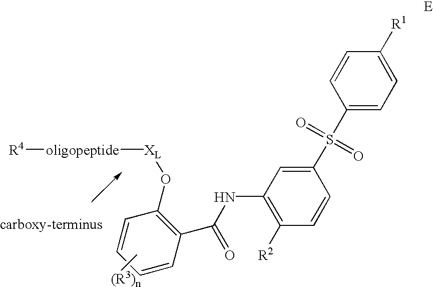
wherein:
oligopeptide is an oligopeptide which is selectively recognized by enzymatically active prostate specific antigen (PSA) and is capable of being proteolytically cleaved by the enzymatic activity of the prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R¹ is halogen;
R² is halogen;
R³ is halogen; and
n is 0, 1 or 2;
R⁴ is selected from
a) hydrogen,
b) —(C═O)R^{a}, 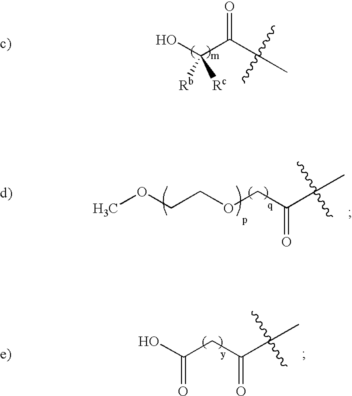
f) ethoxysquarate; and
g) cotininyl;
R^{b} and R^{c} are independently selected from:
a) hydrogen,
b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R^{d}O—, R^{d}C(O)NR^{d}—, (R^{d})₂NC(O)—, R^{d}₂N—C(NR^{d})—, R^{e}S(O)₂NH, CN, NO₂, R^{d}C(O)—, N₃, —N(R^{d})₂, or R^{e}OC(O)NR^{d}—,
c) unsubstituted C₁-C₆ alkyl,
d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R^{d}O—, R^{e}S(O)₂NH, R^{d}C(O)NR^{d}—, (R^{d})₂NC(O)—, R^{d}₂N—C(NR⁶)—, CN, R^{d}C(O)—, N₃, —N(R^{d})₂, and R^{e}OC(O)—NR^{d}—; or
R^{b} and R^{c} are combined to form —(CH₂)^{s}— wherein one of the carbon atoms is optionally replaced by a moiety selected from: O, S(O)ₘ, —NC(O)—, NH and —N(COR^{e})—;
R^{a} is C₁-C₆-alkyl, hydroxylated C₃-C₈-cycloalkyl, polyhydroxylated C₃-C₈-cycloalkyl, hydroxylated aryl, polyhydroxylated aryl or aryl,
R^{d} is selected from: hydrogen, aryl, substituted aryl, heterocycle, substituted heterocycle, C₁-C₆ alkyl and C₃-C₁₀ cycloalkyl;
R^{e} is selected from: aryl, substituted aryl, heterocycle, substituted heterocycle, C₁-C₆ alkyl and C₃-C₁₀ cycloalkyl;
W is selected from a bond, a branched or straight chain C₁-C₆-alkyl, cyclopentyl, cyclohexyl, cycloheptyl or bicyclo[2.2.2]octanyl;
m is 1, 2, 3 or 4;
p is zero or an integer between 1 and 100;
q is 0 or 1, provided that if p is zero, q is 1;
u is selected from: 0, 1, 2 or 3;
y is 1, 2 or 3;
or the pharmaceutically acceptable salt thereof.

12. A conjugate selected from: 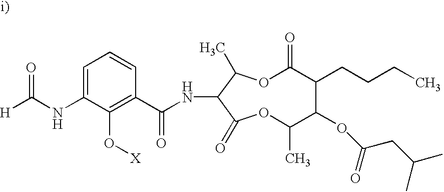
wherein X is 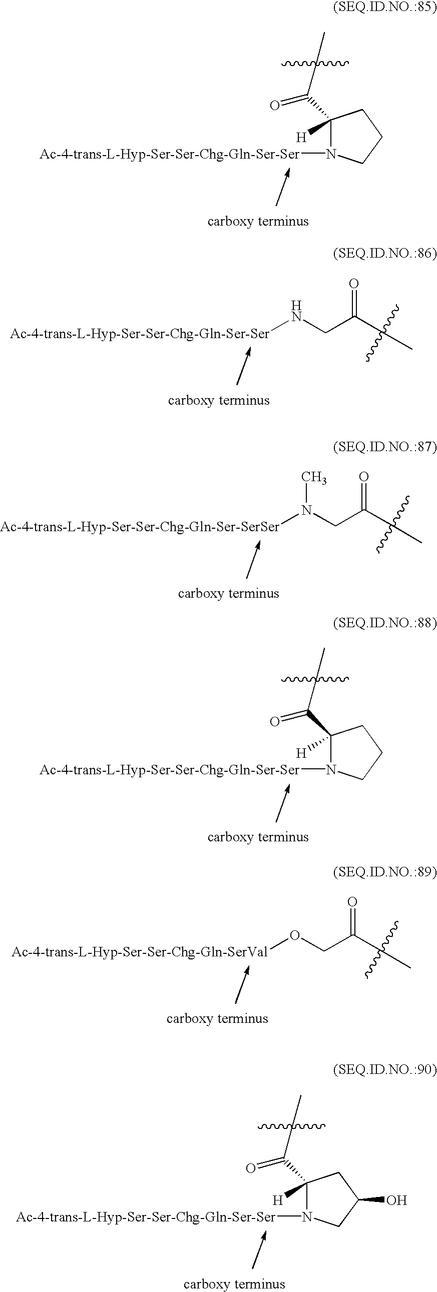 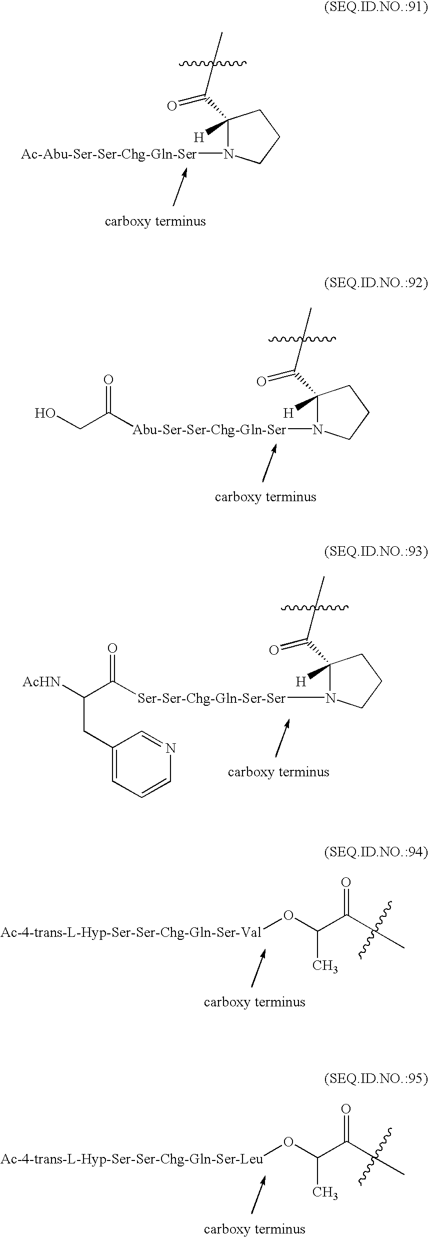 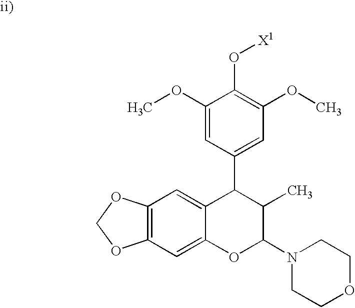
wherein X¹ is 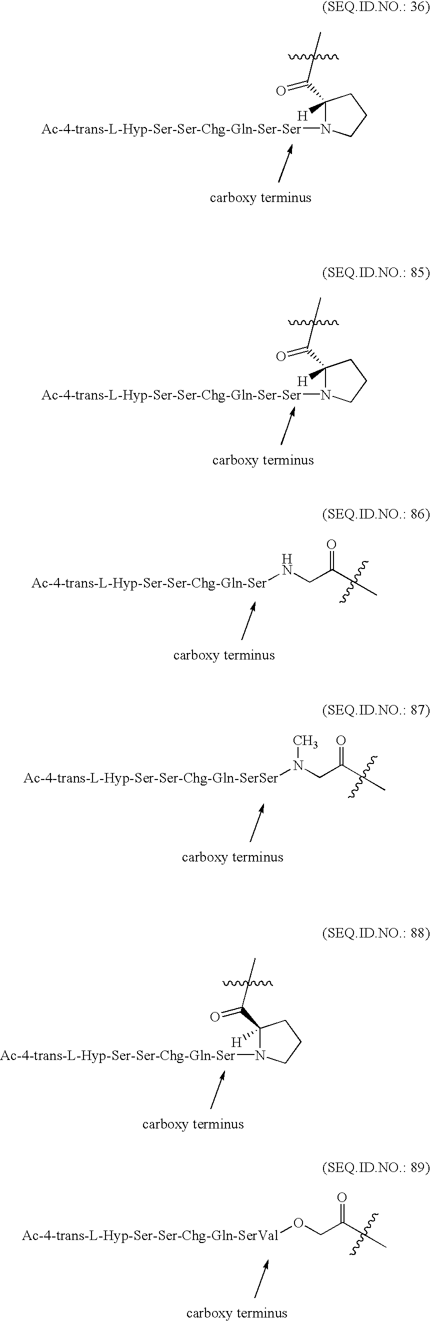 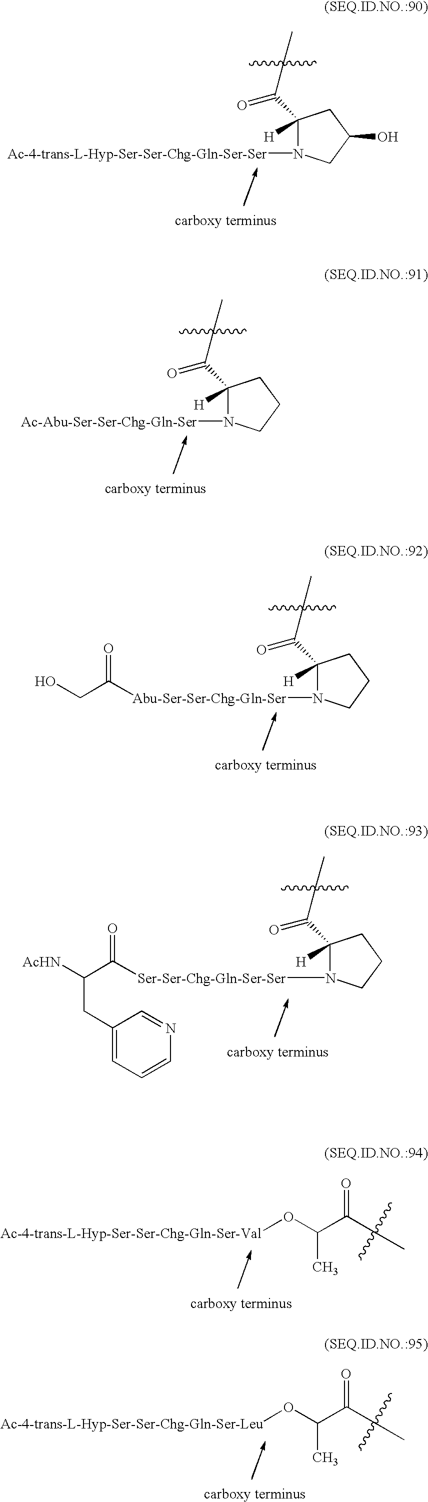 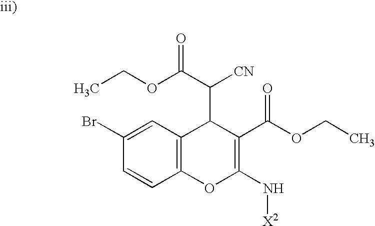
wherein X² is 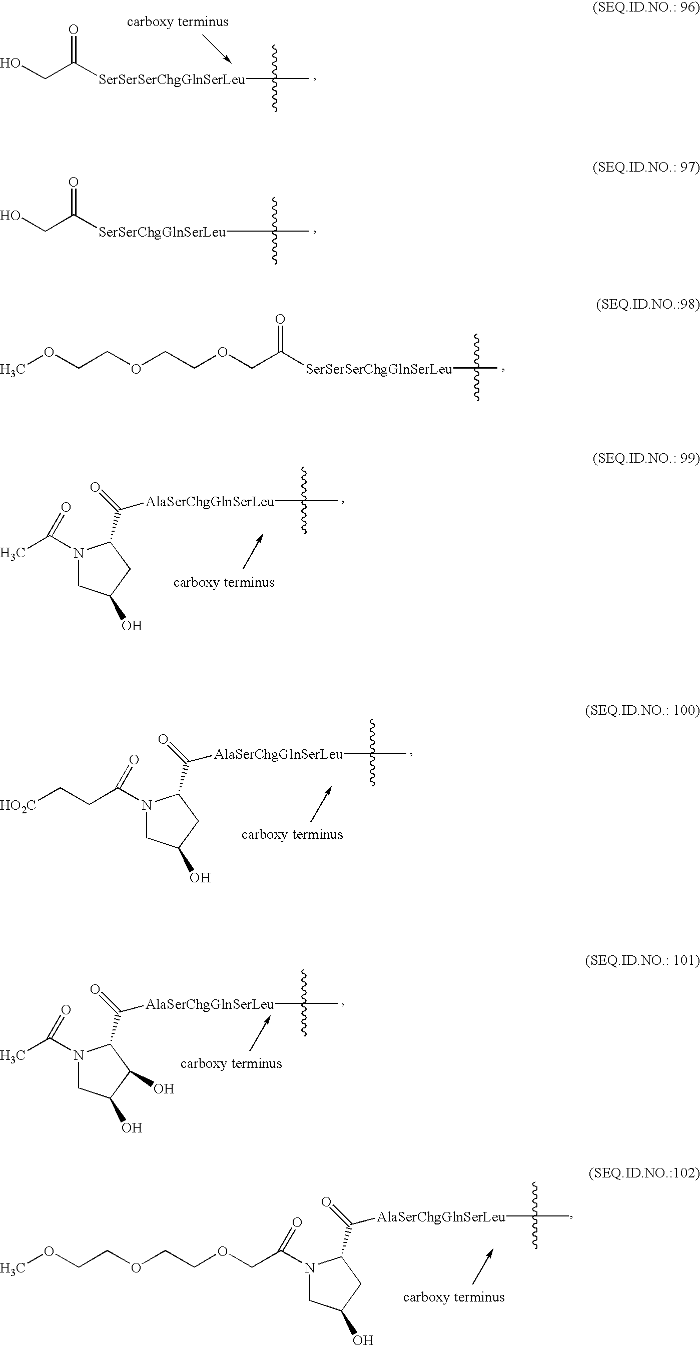 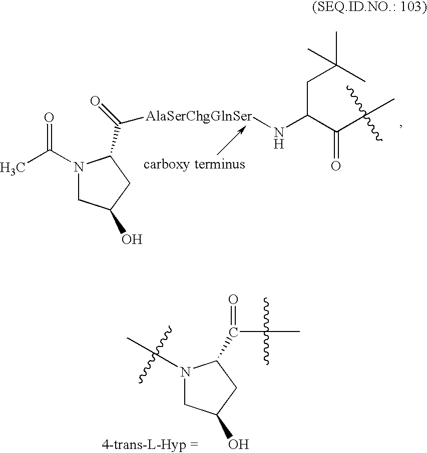 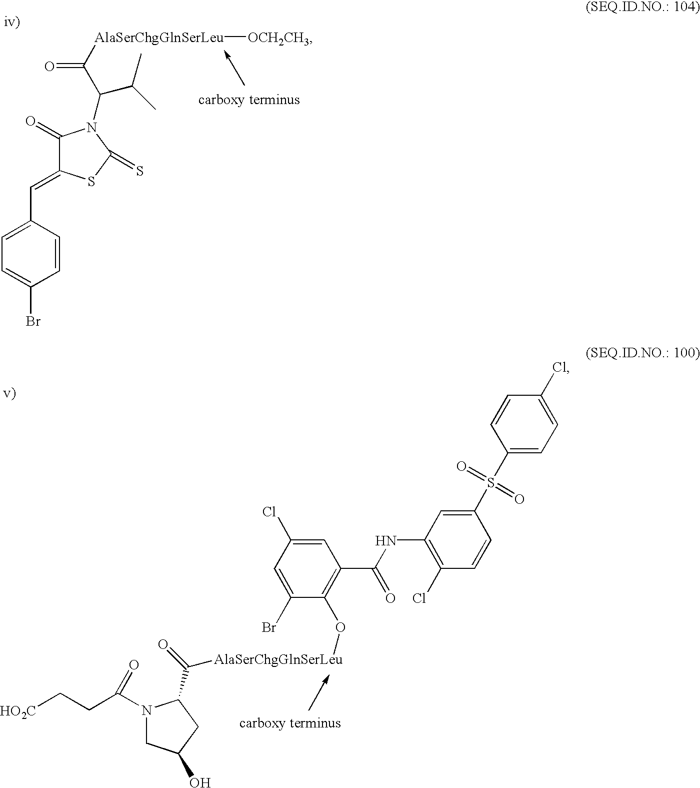
or a pharmaceutically acceptable salt or optical isomer thereof.

13. A pharmaceutical composition comprising a pharmaceutical carrier, and dispersed therein, a therapeutically effective amount of a compound of claim 1.

14. A pharmaceutical composition comprising a pharmaceutical carrier, and dispersed therein, a therapeutically effective amount of a compound of claim 5.

15. A pharmaceutical composition comprising a pharmaceutical carrier, and dispersed therein, a therapeutically effective amount of a compound of claim 10.

16. A method for treating prostate cancer which comprises administering to a mammal in need thereof a therapeutically effective amount of a composition of claim 13.

17. A method for treating prostate cancer which comprises administering to a mammal in need thereof a therapeutically effective amount of a composition of claim 14.

18. A method for treating prostate cancer which comprises administering to a mammal in need thereof a therapeutically effective amount of a composition of claim 15.


SEQUENCE LISTING (108 sequences)

SEQ ID NO: 1 - protein, 7 aa, Artificial Sequence
  Completely Synthetic Amino Acid Sequence
NKISYQS

SEQ ID NO: 2 - protein, 6 aa, Artificial Sequence
  Completely Synthetic Amino Acid Sequence
KISYQS

SEQ ID NO: 3 - protein, 7 aa, Artificial Sequence
  Completely Synthetic Amino Acid Sequence
NKISYYS

SEQ ID NO: 4 - protein, 7 aa, Artificial Sequence
  Completely Synthetic Amino Acid Sequence
NKASYQS

SEQ ID NO: 5 - protein, 5 aa, Artificial Sequence
  Completely Synthetic Amino Acid Sequence
SYQSS

SEQ ID NO: 6 - protein, 5 aa, Artificial Sequence
  Completely Synthetic Amino Acid Sequence
KYQSS

SEQ ID NO: 7 - protein, 5 aa, Artificial Sequence
  Completely Synthetic Amino Acid Sequence
XYQSS

SEQ ID NO: 8 - protein, 7 aa, Artificial Sequence
  Completely Synthetic Amino Acid Sequence
XCHAQSS

SEQ ID NO: 9 - protein, 4 aa, Artificial Sequence
  Completely Synthetic Amino Acid Sequence
YQSS

SEQ ID NO: 10 - protein, 4 aa, Artificial Sequence
  Completely Synthetic Amino Acid Sequence
YQSL

SEQ ID NO: 11 - protein, 4 aa, Artificial Sequence
  Completely Synthetic Amino Acid Sequence
YQSX

SEQ ID NO: 12 - protein, 4 aa, Artificial Sequence
  Completely Synthetic Amino Acid Sequence
XQSX

SEQ ID NO: 13 - protein, 4 aa, Artificial Sequence
  Completely Synthetic Amino Acid Sequence
XQSX

SEQ ID NO: 14 - protein, 4 aa, Artificial Sequence
  Completely Synthetic Amino Acid Sequence
SYQS

SEQ ID NO: 15 - protein, 4 aa, Artificial Sequence
  Completely Synthetic Amino Acid Sequence
SXQS

SEQ ID NO: 16 - protein, 5 aa, Artificial Sequence
  Completely Synthetic Amino Acid Sequence
SYQSV

SEQ ID NO: 17 - protein, 5 aa, Artificial Sequence
  Completely Synthetic Amino Acid Sequence
SXQSV

SEQ ID NO: 18 - protein, 5 aa, Artificial Sequence
  Completely Synthetic Amino Acid Sequence
SYQSL

SEQ ID NO: 19 - protein, 7 aa, Artificial Sequence
  Completely Synthetic Amino Acid Sequence
SCHGQSL

SEQ ID NO: 20 - protein, 6 aa, Artificial Sequence
  Completely Synthetic Amino Acid Sequence
XXSYQS

SEQ ID NO: 21 - protein, 6 aa, Artificial Sequence
  Completely Synthetic Amino Acid Sequence
XXKYQS

SEQ ID NO: 22 - protein, 6 aa, Artificial Sequence
  Completely Synthetic Amino Acid Sequence
XXXYQS

SEQ ID NO: 23 - protein, 6 aa, Artificial Sequence
  Completely Synthetic Amino Acid Sequence
XXXXQS

SEQ ID NO: 24 - protein, 4 aa, Artificial Sequence
  Completely Synthetic Amino Acid Sequence
XYQS

SEQ ID NO: 25 - protein, 6 aa, Artificial Sequence
  Completely Synthetic Amino Acid Sequence
XXSXQS

SEQ ID NO: 26 - protein, 4 aa, Artificial Sequence
  Completely Synthetic Amino Acid Sequence
XXQS

SEQ ID NO: 27 - protein, 5 aa, Artificial Sequence
  Completely Synthetic Amino Acid Sequence
SXQSS

SEQ ID NO: 28 - protein, 5 aa, Artificial Sequence
  Completely Synthetic Amino Acid Sequence
SXQSP

SEQ ID NO: 29 - protein, 5 aa, Artificial Sequence
  Completely Synthetic Amino Acid Sequence
SXQSX

SEQ ID NO: 30 - protein, 6 aa, Artificial Sequence
  Completely Synthetic Amino Acid Sequence
SSYQSV

SEQ ID NO: 31 - protein, 6 aa, Artificial Sequence
  Completely Synthetic Amino Acid Sequence
SSXQSV

SEQ ID NO: 32 - protein, 6 aa, Artificial Sequence
  Completely Synthetic Amino Acid Sequence
SSYQSL

SEQ ID NO: 33 - protein, 6 aa, Artificial Sequence
  Completely Synthetic Amino Acid Sequence
SSXQSL

SEQ ID NO: 34 - protein, 6 aa, Artificial Sequence
  Completely Synthetic Amino Acid Sequence
SSYQSS

SEQ ID NO: 35 - protein, 6 aa, Artificial Sequence
  Completely Synthetic Amino Acid Sequence
SSXQSS

SEQ ID NO: 36 - protein, 6 aa, Artificial Sequence
  Completely Synthetic Amino Acid Sequence
SSYQSP

SEQ ID NO: 37 - protein, 6 aa, Artificial Sequence
  Completely Synthetic Amino Acid Sequence
SSXQSP

SEQ ID NO: 38 - protein, 6 aa, Artificial Sequence
  Completely Synthetic Amino Acid Sequence
XSSYQS

SEQ ID NO: 39 - protein, 6 aa, Artificial Sequence
  Completely Synthetic Amino Acid Sequence
XSSXQS

SEQ ID NO: 40 - protein, 6 aa, Artificial Sequence
  Completely Synthetic Amino Acid Sequence
ASYQSV

SEQ ID NO: 41 - protein, 6 aa, Artificial Sequence
  Completely Synthetic Amino Acid Sequence
ASXQSV

SEQ ID NO: 42 - protein, 6 aa, Artificial Sequence
  Completely Synthetic Amino Acid Sequence
ASYQSL

SEQ ID NO: 43 - protein, 6 aa, Artificial Sequence
  Completely Synthetic Amino Acid Sequence
ASXQSL

SEQ ID NO: 44 - protein, 6 aa, Artificial Sequence
  Completely Synthetic Amino Acid Sequence
XASYQS

SEQ ID NO: 45 - protein, 6 aa, Artificial Sequence
  Completely Synthetic Amino Acid Sequence
XASXQS

SEQ ID NO: 46 - protein, 6 aa, Artificial Sequence
  Completely Synthetic Amino Acid Sequence
SSXQSL

SEQ ID NO: 47 - protein, 6 aa, Artificial Sequence
  Completely Synthetic Amino Acid Sequence
SSXQSV

SEQ ID NO: 48 - protein, 6 aa, Artificial Sequence
  Completely Synthetic Amino Acid Sequence
SSXQSP

SEQ ID NO: 49 - protein, 6 aa, Artificial Sequence
  Completely Synthetic Amino Acid Sequence
SSXQSS

SEQ ID NO: 50 - protein, 7 aa, Artificial Sequence
  Completely Synthetic Amino Acid Sequence
SSSXQSL

SEQ ID NO: 51 - protein, 7 aa, Artificial Sequence
  Completely Synthetic Amino Acid Sequence
SSSXQSV

SEQ ID NO: 52 - protein, 7 aa, Artificial Sequence
  Completely Synthetic Amino Acid Sequence
SSSXQSP

SEQ ID NO: 53 - protein, 7 aa, Artificial Sequence
  Completely Synthetic Amino Acid Sequence
SSSXQSS

SEQ ID NO: 54 - protein, 7 aa, Artificial Sequence
  Completely Synthetic Amino Acid Sequence
SASXQSL

SEQ ID NO: 55 - protein, 7 aa, Artificial Sequence
  Completely Synthetic Amino Acid Sequence
SASXQSV

SEQ ID NO: 56 - protein, 7 aa, Artificial Sequence
  Completely Synthetic Amino Acid Sequence
XSSXQSL

SEQ ID NO: 57 - protein, 7 aa, Artificial Sequence
  Completely Synthetic Amino Acid Sequence
XSSXQSV

SEQ ID NO: 58 - protein, 7 aa, Artificial Sequence
  Completely Synthetic Amino Acid Sequence
XSSYQSV

SEQ ID NO: 59 - protein, 7 aa, Artificial Sequence
  Completely Synthetic Amino Acid Sequence
XSSYQSL

SEQ ID NO: 60 - protein, 7 aa, Artificial Sequence
  Completely Synthetic Amino Acid Sequence
XSSXQSV

SEQ ID NO: 61 - protein, 7 aa, Artificial Sequence
  Completely Synthetic Amino Acid Sequence
XSSXQSL

SEQ ID NO: 62 - protein, 7 aa, Artificial Sequence
  Completely Synthetic Amino Acid Sequence
XSSXQSS

SEQ ID NO: 63 - protein, 7 aa, Artificial Sequence
  Completely Synthetic Amino Acid Sequence
XSSXQSS

SEQ ID NO: 64 - protein, 7 aa, Artificial Sequence
  Completely Synthetic Amino Acid Sequence
XSSXQSP

SEQ ID NO: 65 - protein, 7 aa, Artificial Sequence
  Completely Synthetic Amino Acid Sequence
XSSXQSP

SEQ ID NO: 66 - protein, 7 aa, Artificial Sequence
  Completely Synthetic Amino Acid Sequence
XASXQSV

SEQ ID NO: 67 - protein, 7 aa, Artificial Sequence
  Completely Synthetic Amino Acid Sequence
XASXQSL

SEQ ID NO: 68 - protein, 7 aa, Artificial Sequence
  Completely Synthetic Amino Acid Sequence
XSSYQSV

SEQ ID NO: 69 - protein, 7 aa, Artificial Sequence
  Completely Synthetic Amino Acid Sequence
XSSYQSL

SEQ ID NO: 70 - protein, 7 aa, Artificial Sequence
  Completely Synthetic Amino Acid Sequence
XSAXQSL

SEQ ID NO: 71 - protein, 7 aa, Artificial Sequence
  Completely Synthetic Amino Acid Sequence
XSXXQSL

SEQ ID NO: 72 - protein, 5 aa, Artificial Sequence
  Completely Synthetic Amino Acid Sequence
XXQSL

SEQ ID NO: 73 - protein, 7 aa, Artificial Sequence
  Completely Synthetic Amino Acid Sequence
NRISYQS

SEQ ID NO: 74 - protein, 7 aa, Artificial Sequence
  Completely Synthetic Amino Acid Sequence
NKVSYQS

SEQ ID NO: 75 - protein, 8 aa, Artificial Sequence
  Completely Synthetic Amino Acid Sequence
NKMSYQSS

SEQ ID NO: 76 - protein, 8 aa, Artificial Sequence
  Completely Synthetic Amino Acid Sequence
NKLSYQSS

SEQ ID NO: 77 - protein, 7 aa, Artificial Sequence
  Completely Synthetic Amino Acid Sequence
NKISYQS

SEQ ID NO: 78 - protein, 8 aa, Artificial Sequence
  Completely Synthetic Amino Acid Sequence
QKISYQSS

SEQ ID NO: 79 - protein, 7 aa, Artificial Sequence
  Completely Synthetic Amino Acid Sequence
NXISYQS

SEQ ID NO: 80 - protein, 7 aa, Artificial Sequence
  Completely Synthetic Amino Acid Sequence
NXVSYQS

SEQ ID NO: 81 - protein, 7 aa, Artificial Sequence
  Completely Synthetic Amino Acid Sequence
XASYQSS

SEQ ID NO: 82 - protein, 7 aa, Artificial Sequence
  Completely Synthetic Amino Acid Sequence
XASYQSS

SEQ ID NO: 83 - protein, 5 aa, Artificial Sequence
  Completely Synthetic Amino Acid Sequence
XSXQS

SEQ ID NO: 84 - protein, 7 aa, Artificial Sequence
  Completely Synthetic Amino Acid Sequence
XASXQSS

SEQ ID NO: 85 - protein, 8 aa, Artificial Sequence
  Completely Synthetic Amino Acid Sequence
XASXQSSP

SEQ ID NO: 86 - protein, 8 aa, Artificial Sequence
  Completely Synthetic Amino Acid Sequence
XASXQSSG

SEQ ID NO: 87 - protein, 8 aa, Artificial Sequence
  Completely Synthetic Amino Acid Sequence
XASXQSSX

SEQ ID NO: 88 - protein, 8 aa, Artificial Sequence
  Completely Synthetic Amino Acid Sequence
XASXQSSP

SEQ ID NO: 89 - protein, 7 aa, Artificial Sequence
  Completely Synthetic Amino Acid Sequence
XASXQSX

SEQ ID NO: 90 - protein, 8 aa, Artificial Sequence
  Completely Synthetic Amino Acid Sequence
XASXQSSX

SEQ ID NO: 91 - protein, 7 aa, Artificial Sequence
  Completely Synthetic Amino Acid Sequence
XASXQSP

SEQ ID NO: 92 - protein, 7 aa, Artificial Sequence
  Completely Synthetic Amino Acid Sequence
XASXQSP

SEQ ID NO: 93 - protein, 8 aa, Artificial Sequence
  Completely Synthetic Amino Acid Sequence
XSSXQSSP

SEQ ID NO: 94 - protein, 7 aa, Artificial Sequence
  Completely Synthetic Amino Acid Sequence
XSSXQSX

SEQ ID NO: 95 - protein, 7 aa, Artificial Sequence
  Completely Synthetic Amino Acid Sequence
XSSXQSX

SEQ ID NO: 96 - protein, 7 aa, Artificial Sequence
  Completely Synthetic Amino Acid Sequence
XSSXQSL

SEQ ID NO: 97 - protein, 6 aa, Artificial Sequence
  Completely Synthetic Amino Acid Sequence
XSXQSL

SEQ ID NO: 98 - protein, 7 aa, Artificial Sequence
  Completely Synthetic Amino Acid Sequence
XSSXQSL

SEQ ID NO: 99 - protein, 7 aa, Artificial Sequence
  Completely Synthetic Amino Acid Sequence
XASXQSL

SEQ ID NO: 100 - protein, 7 aa, Artificial Sequence
  Completely Synthetic Amino Acid Sequence
XASXQSL

SEQ ID NO: 101 - protein, 7 aa, Artificial Sequence
  Completely Synthetic Amino Acid Sequence
XASXQSL

SEQ ID NO: 102 - protein, 7 aa, Artificial Sequence
  Completely Synthetic Amino Acid Sequence
XASXQSL

SEQ ID NO: 103 - protein, 7 aa, Artificial Sequence
  Completely Synthetic Amino Acid Sequence
XASXQSX

SEQ ID NO: 104 - protein, 6 aa, Artificial Sequence
  Completely Synthetic Amino Acid Sequence
ASXQSX

SEQ ID NO: 105 - protein, 7 aa, Artificial Sequence
  Completely Synthetic Amino Acid Sequence
XASXQSX

SEQ ID NO: 106 - protein, 7 aa, Artificial Sequence
  Completely Synthetic Amino Acid Sequence
XASXQSL

SEQ ID NO: 107 - protein, 4 aa, Artificial Sequence
  Completely Synthetic Amino Acid Sequence
XQSX

SEQ ID NO: 108 - protein, 4 aa, Artificial Sequence
  Completely Synthetic Amino Acid Sequence
XQSX